(19) United States
(12) Patent Application Publication
(10) Pub. No.: US 2013/0136713 A1
(43) Pub. Date: May 30, 2013

(54) DEODORANT COMPOSITION FOR SULFIDES

(71) Applicants: Ikuo Terada, Kanagawa (JP); Sadahiko Yamazaki, Kanagawa (JP); Kunihide Hoshino, Kanagawa (JP); Mitsuhiko Fujiwhara, Kanagawa (JP)

(72) Inventors: Ikuo Terada, Kanagawa (JP); Sadahiko Yamazaki, Kanagawa (JP); Kunihide Hoshino, Kanagawa (JP); Mitsuhiko Fujiwhara, Kanagawa (JP)

(73) Assignee: TAKASAGO INTERNATIONAL CORPORATION, Tokyo (JP)

(21) Appl. No.: 13/814,967

(22) PCT Filed: Sep. 21, 2011
(86) PCT No.: PCT/JP2011/072685

§ 371 Date: Feb. 8, 2013

(30) Foreign Application Priority Data
Sep. 24, 2010 (JP) 2010-214196

(51) Int. Cl. A61L 9/01

(52) U.S. Cl. CPC A61L 9/01
USPC 424/76.1

(57) ABSTRACT

The present invention provides a deodorant composition which specifically acts upon a malodor derived from sulfides and thereby can eliminate or alleviate the malodor. The present invention relates to a deodorant composition for sulfides, which comprises (A) a polyphenol or a polymer thereof and (B) sodium percarbonate or hydrogen peroxide.

### TECHNICAL FIELD

The present invention relates to a deodorant composition for sulfides. More illustratively, it relates to a deodorant composition for sulfides, which specifically acts upon sulfides as the source of malodor and thereby eliminates or alleviates the malodor.

### BACKGROUND ART

In recent years, attention has been paid to various points around ourselves with the divergence of life, improvement of life style, change and improvement of consciousness and the like. One of them is the presence of various malodors. As the main components of the malodors, nitrogen-containing compounds such as ammonia, urea, indole, skatole and amines, sulfur-containing compounds such as methyl mercaptan, hydrogen sulfide and dimethyl sulfide, lower fatty acids such as butyric acid, and the like are known. There are a large number of reports on deodorants to be used in eliminating or alleviating these malodors.

For example, there is a report in which a plant extract containing a mixture of various polyphenol compounds is used as a deodorant (e.g., see Patent Reference 1). The deodorant shows a deodorization effect to some degree but there are many cases where it cannot be said that sufficient deodorization effect is shown, so that there is room for further improvement regarding this point.

As a deodorant composition which had improved this point, there is a report in which a deodorant composition which uses, as the active ingredient, a colored compound obtained by allowing a polyphenol compound to undergo the reaction at a pH value of 6.5 or more in a solvent showing alkalinity in the coexistence of an oxygen molecule (cf. see Patent Reference 2). It is described that, in the deodorant composition, its deodorizing ability is kept even after the lapse of prolonged period of time once the deodorant composition is prepared, and moreover, it can possess its excellent deodorization effect particularly for malodor components such as nitrogen-containing compounds and sulfur-containing compounds.

### CITATION LIST

### Patent Literature

PTL 1: JP-A-11-319051
PTL 2: JP-A-2004-167218

### SUMMARY OF INVENTION

### Technical Problem

However, the deodorization effect of these deodorant compositions for sulfides is not sufficient.

Accordingly, a purpose of the invention is to provide a deodorant composition which specifically acts upon malodors particularly derived from sulfides and thereby can eliminate or alleviate the malodors.

### Solution to Problem

In order to solve the above-mentioned problems, the inventors have conducted intensive studies and found as a result that the deodorization effect for sulfides is improved when a polyphenol compound is combined with a specific oxidant, thus resulting in the accomplishment of the invention.

That is, the problem of the invention can be solved by the following (1) to (6).

(1) A deodorant composition for sulfides, which comprises (A) a polyphenol or a polymer thereof and (B) sodium percarbonate or hydrogen peroxide.

(2) The deodorant composition for sulfides according to (1), wherein the polymer of the polyphenol is a compound having a molecular weight of 10,000 or less which is obtained by allowing the polyphenol to undergo a reaction in an alkaline solvent.

(3) The deodorant composition for sulfides according to (1) or (2), wherein a mixing ratio ((A):(B)) of the component (A) to the component (B) is from 1:0.1 to 1:1000 in terms of mass ratio.

(4) The deodorant composition for sulfides according to any one of (1) to (3), wherein the component (A) is the polymer of the polyphenol.

(5) The deodorant composition for sulfides according to (4), wherein the polymer of the polyphenol is a compound obtained by allowing a tea extract to undergo a reaction in an alkaline solvent.

(6) The deodorant composition for sulfides according to any one of (1) to (4), wherein the polyphenol is at least one species selected from the group consisting of a raw coffee bean extract, a tea extract and gallic acid.

### Advantageous Effects of Invention

According to the invention, a deodorant composition which has excellent deodorization effect for malodor components, particularly the malodor components derived from sulfides such as dimethyl sulfide, dimethyl disulfide and dimethyl trisulfide, can be provided. Accordingly, since it can produce excellent deodorization effect upon the malodor components derived from sulfides, for example, in toilet room, combustible trash, body odor and the like, the malodors which are felt in a daily life can be eliminated or alleviated.

### DESCRIPTION OF EMBODIMENTS

The following describes the invention in detail.

The deodorant composition for sulfides of the invention comprises (A) a polyphenol or a polymer thereof and (B) sodium percarbonate or hydrogen peroxide.

### <(A) Polyphenol or a Polymer Thereof>

Firstly, the polyphenol as the material for preparing the deodorant composition for sulfides of the invention is described. The polyphenol to be used in the invention means a compound in which two or more hydroxyl groups are substituted with hydrogen atoms on one benzene ring, and its glycoside is also included as a polyphenol. The polyphenol to be used in the invention is not particularly limited, but a polyphenol having hydroquinone or o-diphenol structure is desirable. In this connection, the o-diphenol structure means a structure when hydroxyl groups are directly substituted on the benzene ring and the hydroxyl groups are adjoined each other.

Specific examples of the polyphenol include apigenin, apigenin glycosides, acacetin, isorhamnetin, isorhamnetin glycosides, isoquercitrin, epicatechin, epicatechin gallate, epigallocatechin, epigallocatechin gallate, aesculetin, ethyl protocatechuate salt, ellagic acid, catechol, γ-acid, catechin, gardenin, gallocatechin, caffeic acid, caffeic esters, chlorogenic acid, kaempferol, kaempferol glycosides, quercetin, quercetin glycosides, quercetagenin, genistin, genistin glycoside, gossypetin, gossypetin glycosides, gossypol, 4-dihydroxyanthraquinone, 1,4-dihydroxynaphthalene, cyanidin, cyanidin glycosides, sinensetin, diosmetin, diosmetin glycosides, 3,4′-diphenyldiol, sinapic acid, stearyl-β-(3,5-di-tert-butyl-4-hydroxyphenyl)propionate, spinacene, tangeritin, taxifolin, tannic acid, daphnetin, tyrosine, delphinidin, delphinidin glycosides, theaflavine, theaflavine monogallate, theaflavine bisgallate, tricetinidin, dopa, dopamine, naringenin, naringin, nordihydroguairetic acid, noradrenaline, hydroquinone, vanillin, patchouletin, herbacetin, vanillyl alcohol, vanitrope, vanillin propylene glycol acetal, vanillic acid, bis(4-hydroxyphenyl)sulfonic acid, bisphenol A, pyrocatechol, vitexin, 4,4′-biphenyldiol, 4-tert-butylcatechol, 2-tert-butylhydroquinone, protocatechuic acid, phloroglucinol, phenolic resins, procyanidin, prodelphinidin, phloretin, phloretin glycosides, fisetin, folin, fervasetin, fraxetin, phloridzin, paeonidin, paeonidin glycosides, pelargonidin, pelagugonidin glycosides, petunidin, petunidin glycosides, hesperetin, hesperidin, gallic acid, gallic esters (lauryl gallate, propyl gallate, butyl gallate), manjiferin, malvidin, malvidin glycosides, myricetin, myricetin glycosides, 2,2′-methylenebis(4-methyl-6-tert-butylphenol), 2,2′-methylenebis(4-ethyl-6-tert-butylphenol), 2,2′-methylenebis(4-methyl-6-tert-butylphenol), 2,2′-methylenebis(4-ethyl-6-tert-butylphenol), methyl atrarate, 4-methylcatechol, 5-methylcatechol, 4-methoxycatechol, 5-methoxycatechol, methylcatechol-4-carboxylic acid, 2-methylresorcinol, 5-methylresorcinol, morin, limocitrin, limocitrin glycosides, limocitrol, luteolin, luteolin glycosides, luteolinidin, luteolinidin glycosides, rutin, resorcin, resveratrol, resorcinol, leukocyanidin, leukodelphinidin, and the like. These polyphenols may be used alone or as a mixture of two or more species.

In addition, though the above-mentioned polyphenol can be prepared by a commonly known method, a commercially available product may be purchased. In addition, the polyphenol may be prepared by synthesis. Further, a high concentration polyphenol fraction prepared from a plant can also be used.

Also, according to the invention, a plant extract containing a polyphenol can also be used.

Examples of the plant extract include extracts obtainable from aloe, anise seeds, elder, eleutherococcus, psyllium, orange flower, allspice, oregano, valerian, chamomile, capsicum pepper, cardamon, cassia, garlic, caraway seeds, clove, cumin seeds, kola, coriander seeds, Rhus javanica, saffron, zanthoxylum, juniper berry, cinnamon, ginger, star anise, St. Johns wart, celery seed, savory, sesame, pieplant, tarragon, turmeric, thistle, Anethum graveolens, nutmeg, nettle, hibiscus, hamamelis, birch, basil, bitter orange, fennel, primrose, fenugreek, verbena, Laurus nobilis, hop, boldo, horseradish, poppy seed, gallnut, marigold, marrow, marjoram, mustard, Millefeuille, mint leaves, melissa, mace, lindane, Gentiana scabra var. buergeri, rosehip, rosemary, Rosmarinus officinalis, sunflower seeds, grape pericarp, apple, carrot leaves, banana, strawberry, apricot, peach, plum, pineapple, pear, persimmon, cherry, papaya, mango, avocado, melon, loquat, fig, kiwi, prune, blueberry, black berry, raspberry, cranberry, coffee beans, cacao beans, grape seeds, grape fruits seeds, pecan nut, cashew nut, chestnut, coconut, peanut, walnut, green tea leaves, black tea leaves, oolong tea leaves, tobacco, perilla leaves, garden thyme, sage, lavender, spearmint, peppermint, spotted thistle, hyssop, sweet basil, marigold, dandelion, artichoke, Matricaria chamomille, Agrimonia pilosa var. japonica, licorice, anise, yarrow, eucalyptus, wormwood, balm, Angelica pubescens, fenugreek, Capsicum annuum var. angulosum, fennel, red pepper, coriander seeds, caraway seeds, fennel seeds, ginger, horseradish, Origanum majorana, Origanum vulgare, mustard, parsley, pepper, savory, tarragon, queen lily, wasabi, dill seeds, citrus fruits, and the like. These plant extracts may be used alone or as a mixture of two or more species.

In addition, according to the invention, a polymer prepared from a polyphenol can be used. The polymer of the polyphenol can be obtained by allowing the polyphenol to undergo the reaction in an alkaline solvent.

The solvent showing alkalinity is a known one and is representatively an alkaline substance-containing solvent wherein an alkaline substance is dissolved in a solvent such as water.

The alkaline substance is not particularly limited and specifically includes carbonate salts or hydrogen carbonate salts such as sodium carbonate, potassium carbonate, sodium bicarbonate, ammonium carbonate, and guanidine carbonate; borate salts such as potassium borate and sodium borate; silicate salts such as potassium silicate, sodium silicate No. 1, sodium silicate No. 2, sodium silicate No. 3, sodium orthosilicate, and sodium metasilicate; sodium monohydrogen phosphate, sodium sulfite, sodium hydroxide, calcium hydroxide, potassium hydroxide, magnesium hydroxide, ammonium hydroxide, sodium pyrophosphate, and potassium pyrophosphate; and the like.

The solvent for dissolving one or two or more these alkaline substances includes water and various hydrous solvent as preferable solvents. Moreover, so-called alkaline buffer solutions using these alkaline substances and acids may be also employed.

The above solvent usually show alkalinity and it is alkaline before the reaction but sometimes shows weak acidity depending on the substance to be incorporated into the solvent and an added amount thereof. Namely, when the solvent before the reaction is necessarily alkaline and, at obtaining the above deodorant composition, the pH of the solvent in the reaction system after the start of the reaction is 6.5 or more, then a preferable result is obtained. In particular, the pH of the reaction system is preferably in the range of 7 to 13, further preferably in the range of 8 to 13. When the pH in the reaction system during the reaction is below 6.5, it is impossible to afford a deodorant composition having a preferable deodorizing effect. On the other hand, when the pH is too high (around pH of 14), it requires attention when the deodorant composition is handled, thus is inconvenient.

With regard to the temperature at the reaction, the inventive product can be obtained at a temperature of 0° C. to a reflux temperature of the solvent, but when the reaction is carried out at a high temperature, e.g. 60° C. or more, the amount of dissolved oxygen is largely decreased to dramatically reduce the production efficiency of the deodorizing active component, and in addition thereto, there is a possibility that the produced deodorizing active component is decomposed by heat, and thus the case is not preferable. Thus, the reaction is carried out at a temperature of preferably 0° C. to 60° C., more preferably 0° C. to 40° C., further preferably 0° C. to 25° C., resulting in the efficient production of the deodorizing active component. However, even if the reaction is carried out at a high temperature, e.g. 60° C. or more, the effective deodorant component can be produced by lowering the reaction liquid and supplying oxygen by means of stirring or the like.

In the invention, the polyphenol preferably reacts within a short period of time but from a practical viewpoint, the reaction may be carried out for preparing polyphenol polymer for preferably from several minutes (2 minutes) to about 24 hours, more preferably from about 10 minutes to about 9 hours, further preferably from 10 minutes to 7 hours.

At the reaction for preparing the above polyphenol polymer, pressurization is not particularly necessary but pressure may be applied. In addition, oxygen may present during the reaction, and amino acid may be further contained therein.

The polyphenol polymer prepared in the invention includes a colored compound. This colored compound takes a role as a deodorization active component. It is desirable that molecular weight of the thus obtained polyphenol polymer exceeds molecular weight of the polyphenol as the starting substance which is not subject to the reaction and is 10,000 or less.

According to the invention, the polyphenol or a polymer thereof is not particularly limited, but it is desirable to use gallic acid, a raw coffee bean extract, a tea extract, polymer of these plant extracts, and the like, and it is particularly desirable to use a polymer prepared by allowing the tea extract to undergo the reaction in an alkaline solvent.

### <(B) Sodium Percarbonate and Hydrogen Peroxide>

The deodorant composition for sulfides of the invention comprises sodium percarbonate or hydrogen peroxide as an essential component.

Though details of the reason of obtaining such an excellent effect by the deodorant composition of the invention is not necessarily clear, it is considered that the effect upon sulfides is synergistically improved by mixing sodium percarbonate or hydrogen peroxide with the above-mentioned polyphenol or a polymer thereof. Sodium percarbonate and hydrogen peroxide may be respectively used alone or as a mixture thereof.

### <Preparation of Deodorant Composition>

The mixing ratio ((A):(B)) of the above-mentioned polyphenol or a polymer thereof (component (A)) to the above-mentioned sodium percarbonate or hydrogen peroxide (component (B)) is preferably from 1:0.1 to 1:1,000 (mass ratio), more preferably from 1:1 to 1:100 (mass ratio), further more preferably from 1:5 to 1:50 (mass ratio), in the deodorant composition.

When the mixing ratio of the component (A) to component (B) is within the aforementioned range, the effect of the invention can be properly obtained.

According to the invention, when the reaction is carried out in the coexistence of a metal ion or a metal salt which releases a metal ion into the reaction system, a more excellent deodorant composition exhibiting an enhanced deodorizing activity and stability can be obtained.

As a preferable metal ion, there may be mentioned a copper ion, a zinc ion, a calcium ion, a magnesium ion, a silver ion, a tin ion, an aluminum ion, or a manganese ion.

Examples of the compound which releases a metal ion include the following: e.g., copper compounds such as copper chloride, copper fluoride, copper sulfate, copper nitrate, copper hydroxide, copper citrate, copper gluconate, copper aspartate, copper glutamate, sodium copper chlorophyllin and copper chlorophyll; zinc compounds such as zinc chloride, zinc fluoride, zinc sulfate, zinc nitrate, zinc hydroxide, zinc citrate, zinc gluconate, zinc aspartate, zinc glutamate, zinc phosphate, and zinc lactate; calcium compounds such as calcium chloride, calcium hydroxide, calcium citrate, calcium gluconate, calcium L-glutamate, calcium carbonate, calcium lactate, calcium pantothenate, calcium dihydrogen pyrophosphate, calcium propionate, calcium sulfate, tricalcium phosphate, calcium monohydrogen phosphate, calcium dihydrogen phosphate, and disodium calcium ethylenediaminetetraacetate; magnesium compounds such as magnesium chloride, magnesium sulfate, magnesium hydroxide, magnesium L-glutamate, magnesium oxide, and magnesium carbonate; silver compounds such as silver oxide; tin compounds such as tin chloride, tin acetate, and tin fluoride; aluminum compounds such as aluminum chloride, aluminum hydroxide, aluminum acetate, aluminum borate, aluminum phosphate, and aluminum sulfate; permanganese salts such as potassium permanganese, manganese compounds such as manganese sulfate, and the like. In addition, titanium compounds such as titanium dioxide can be also employed.

The addition amount of the metal ion varies depending on the situation of the reaction but it is preferable to add the ion so that the concentration of the metal ion in the reaction liquid becomes 0.00001 mM to 100 mM and the concentration is more preferably from 0.00005 mM to 10 mM, further preferably from 0.1 mM to 5 mM.

At the preparation of the deodorant composition of the invention, additives already conventionally used may be coexisted in the reaction system.

The thus obtained reaction liquid containing the deodorizing active component can be used as the deodorant composition without further treatment. Alternatively, if necessary, by a method of further concentrating the reaction liquid containing the deodorizing active component or the like method, a deodorant composition having a high content of the deodorizing active component can be obtained. Furthermore, a solid deodorant composition can be obtained by removing liquid components from the reaction liquid containing the deodorizing active component by a known method such as a vacuum-drying method or a freeze-drying method. Alternatively, the liquid may be supported on any carrier such as a liquid, a solid, or a gel substance to form a deodorant composition.

Preferred examples of the liquid include water, hydrous alcohols, lower alcohols (methanol, ethanol, butanol, propanol, etc.), polyol-based organic solvents (ethylene glycol, propylene glycol, etc.), benzyl alcohol, glycerin, monoglycerides, diglycerides, animal and plant oils, essential oils, and the like.

Preferred examples of the solid include porous carriers, e.g., sugars such as dextrin, cyclodextrin, glucose, lactose, and starch; plastic carriers such as plastic particles and foam plastics; inorganic particles such as silica gel particles, diatomaceous earth, activated clay, vermiculite, alumina, zeolite, perlite, clay minerals, unglazed pottery, ceramics, metals, glass, and active carbon; water-absorbable polymers; natural carriers such as buckwheat chaff, chaff, sawdust, and baked products thereof; fibrous carriers such as fibers, fiber aggregates, fiber bunch, non-woven fabrics, knitted goods, textiles, pulp, paper, paper products (cardboards, honeycomb, etc.); synthetic molecules such as crown ethers, cryptands, cyclophanes, and carixarenes; and the like. The “porous” herein includes the case that the carrier itself is porous and the case that numerous voids are present between the carriers.

Examples of the gel substances include aqueous gelating agents such as carrageenin, carboxyvinyl polymers, crosslinked polyacrylic acid, hydroxyethyl cellulose, carboxymethyl cellulose, sodium acrylate, agar, gelatin, pectin, pharselan, xanthan gum, locust bean gum, duran gum, and collagen; oily gelating agents such as metal soaps and dibenzylidene sorbitol. They can be used solely or in combination.

As a method for supporting the deodorant composition of the invention on the carrier, there can be mentioned a method of attaching the deodorant composition in a solution state by means of coating, impregnation, spraying, or the like and subsequently drying (e.g., air-drying at 60° C. for 12 hours) as an example.

The deodorant composition of the invention may be used after capsulation by a known method using gelatin, gum arabic, sodium arginate, a cellulose derivative such as ethyl cellulose, polyvinyl alcohol, vinyl methyl ether-maleic anhydride copolymer, styrene-maleic anhydride copolymer, polyethylene, polystyrene, paraffin wax, or the like.

Moreover, particularly in the case that the deodorant composition of the invention is used in a solution state, the reduction of the amount of dissolved oxygen in the solution as far as possible remarkably enhances the storage stability of the composition of the invention in the solution and thus the case is convenient.

As a method for the reduction of the amount of dissolved oxygen in the solution as far as possible, a known method may be employed, which specifically includes a method of storing the solution under a circumstance of reduced pressure, a method of subjecting the solution to degas treatment, a method of replacement with nitrogen gas or argon gas, a method of treating it under an atmosphere of nitrogen gas or argon gas, and the like.

In the case that the deodorant composition is used in a solid state, when a compound having deliquescence or high hygroscopicity is present together with the deodorant composition, the compound efficiently absorbs the moisture in the air and hence a reaction field suitable for the deodorant composition may be provided, so that the case is more preferred for exhibiting the deodorizing effect of the deodorant composition.

As examples of the compound having deliquescence or high hygroscopicity, salts and alkalis showing deliquescence by the moisture in the air or showing a property of strongly absorbing the moisture in the air are employed and particularly, salts having deliquescence or high hygroscopicity are practical.

Specific examples include lithium chloride, sodium chloride, potassium chloride, magnesium chloride, ammonium magnesium chloride, sodium magnesium chloride, potassium magnesium chloride, manganese chloride, potassium manganese chloride, antimony chloride, antimony cobalt chloride, zinc chloride, iron chloride, bismuth chloride, beryllium chloride, calcium bromide, zinc bromide, copper bromide, iron bromide, cobalt bromide, cadmium bromide, lithium iodide, sodium iodide, magnesium iodide, calcium iodide, iron iodide, nickel iodide, sodium nitrite, potassium nitrite, magnesium nitrite, ammonium nitrate, lithium nitrate, sodium nitrate, calcium nitrate, beryllium nitrate, magnesium nitrate, manganese nitrate, cerium nitrate, ammonium cerium nitrate, iron nitrate, copper nitrate, lithium chlorate, calcium chlorate, magnesium chlorate, zinc chlorate, cadmium chlorate, cobalt chlorate, copper chlorate, potassium carbonate, lithium sulfate, ammonium zinc sulfate, antimony sulfate, iron sulfate, ammonium cadmium sulfate, ammonium thiosulfate, potassium phosphate, ammonium phosphite, potassium phosphite, hydrazinium phosphite, sodium hypophosphite, potassium hypophosphite, sodium permanganate, calcium permanganate, strontium permanganate, magnesium permanganate, zinc permanganate, sodium hydroxide, potassium hydroxide, and the like. These salts may be used solely or two or more of them may be used in combination.

The most suitable amount of the compound having deliquescence or high hygroscopicity to be co-present varies depending on the kind of the compound, the environment to be applied, and intended use and hence the amount is not unconditionally determined but 0.1 to 10 weight equivalents to the deodorant composition may be mentioned as an example.

In the invention, various additives commercially available can be added to the deodorant composition obtained by the above method. As the additives, for example, there may be mentioned an extender, an antioxidant, a dyestuff, a known deodorant, a surfactant, a flavor or fragrance, a stabilizer, an antibacterial agent, a moisture absorbent (calcium chloride, a high water absorbing polymer or the like), a filler (lactose or the like), an antifoaming agent and the like.

They can be mixed with the deodorant composition of the invention solely or as a combination of two or more of them and thus a characteristic deodorant composition and deodorant can be prepared. Particularly, when an antibacterial agent is mixed to the deodorant composition, the deodorizing effect is synergistically enhanced and hence it becomes possible to prepare a more characteristic deodorant composition and deodorant by combining the agent with other additives to exhibit performances of the additives. The mixing amounts of the above additives are not particularly limited as far as the amounts are such amounts that they can accomplish desired purposes.

The extenders include sugars, polysaccharides, processed starch, casein, gelatin, carboxymethyl cellulose, lecithin, and the like.

As the antioxidants, there are known butylhydroxytoluene, butylhydroxyanisole, citric acid, biofavoic acid, glutathione, selenium, licopene, vitamin A, vitamin E, and vitamin C, and also pyrrolopyrrole derivatives, free radical scavengers obtainable form extracts of various plants, enzymes having antioxidant properties such as superoxide dismutases and glutathione peroxidases, and the like.

As the dyestuffs, dyes, lakes, organic synthetic dyestuffs (tar dyestuffs) such as organic pigments, natural dyestuffs, inorganic pigments, and the like are known. Specifically, there are known hibiscus dyestuff, huckleberry dyestuff, plume dyestuff, layer dyestuff, duberry dyestuff, grape juice dyestuff, blackberry dyestuff, blueberry dyestuff, mulberry dyestuff, morello cherry dyestuff, red currant dyestuff, loganberry dyestuff, paplica powder, malt extract, rutin, flavonoids, red cabbage dyestuff, red radish dyestuff, adzuki bean dyestuff, turmeric dyestuff, olive tea, cowberry dyestuff, chlorella dyestuff, saffron dyestuff, perilla dyestuff, strawberry dyestuff, chicory dyestuff, pecannut dyestuff, red rice malt dyestuff, safflower dyestuff, purple sweet potato dyestuff, lac dyestuff, spirulina dyestuff, onion dyestuff, tamarind dyestuff, chili pepper dyestuff, gardenia dyestuff, Gardenia jasminoides dyestuff, sikon dyestuff, rosewood dyestuff, euphausiid dyestuff, orange dyestuff, carrot carotene, carmel, sodium iron chlorophyllin, riboflavin, norbixin potassium, norbixin sodium, alamance, erythrocin, new coccin, phloxine B, rose bengal, acid red, cutoradin, sunset yellow, first green, brilliant blue, indigocarmine, lake red C, lithol red, rhodamine, phloxine, indigo, ponceau, orange I, sudan blue, and the like. Inorganic pigments include mica, talc, calcium carbonate, kaolin, silicic anhydride, aluminum oxide, colcothar, iron oxide, ultramarine, carbon black, titanium dioxide, zinc dioxide, mica, bismuth oxychloride, boron nitride, photochromic pigments, hybrid fine powder, synthetic mica, and the like.

The antibacterial agents include benzoic acid, sodium benzoate, isopropyl p-hydroxybenzoate, isobutyl p-hydroxybenzoate, ethyl p-hydroxybenzoate, methyl p-hydroxybenzoate, butyl p-hydroxybenzoate, propyl p-hydroxybenzoate, sodium sulfite, sodium hyposulfite, potassium pyrosulfite, sorbic acid, potassium sorbate, sodium dehydroacetate, thujaplicin, udo extract, storax extract, wild tansy extract, milt protein extract, zymolytic Job's tears extract, and the like.

Examples of known deodorants include deodorants due to the desulfurizing action of iron sulfates such as ferrous sulfate and iron chlorides; deodorants due to the chemical reaction of acidic agents, alkaline agents, oxidizing agents, decoloring agent, ozone and the like; deodorants due to the adding or condensing action of (meth)acrylate esters, maleate esters, and the like as adding agents or glyoxal as a condensing agent; deodorants due to the ion-exchanging action of amphoteric ion-exchange resins, cationic ion-exchange resins, anionic ion-exchange resins, and the like; deodorants due to the chemical substance-attaching or adsorbing action of alkaline or acidic attaching active carbon, mixtures of active carbon and a chemical reagent, and the like; deodorants due to the adsorbing action of porous adsorbents such as neutral active carbon, a fibrous carbon deodorant, zeolite, and active clay; deodorants due to the enzymatic action of digestive enzymes or enzymes produced by mouth good bacterium LS-1 lactic acid bacterium, yeasts, soil bacteria, and the like or bacteria thereof; deodorants due to the antiseptic or bactericidal action of chloramine T, parabens, phenols, and the like; polyphenol deodorants such as persimmon polyphenol, tea chatechin, rosemary extract, bamboo extract, oolong tea extract, black tea extract, green tea extract, tansy extract, white oak leave extract, and rice bran/soy bean-firing extract; and the like. In addition, there are also included cyclodextrin, champignon extract, rooibos extract, sodium iron chlorophyllin, active carbon, zeolite, and the like.

The surfactants include nonion types (polyoxyethylene alkyl ethers, fatty acid alkylolamides, etc.), acylglutamic acid types, and the like. These surfactants are preferably used solely or as a combination of two or more of them. Examples of the polyoxyethylene alkyl ethers include polyoxyethylene stearyl, polyoxyethylene hardened castor oil, and the like. Examples of the fatty acid alkylolamides include coconut-oil fatty acid diethanolamide. The acylglutamic acid types include glutamate esters of saturated and unsaturated fatty acids having 12 to 18 carbon atoms and coconut-oil fatty acids, hardened coconut-oil fatty acids, palm-oil fatty acids, hardened palm oil fatty acids, beef-tallow fatty acids, hardened beef-tallow fatty acids, and the like which are mixtures thereof, and specifically, include N-coconut-oil fatty acid-acyl-L-glutamic triethanolamine, lauroyl-L-glutamic triethanolamine, sodium N-coconut-oil fatty acid-acyl-L-glutamate, sodium N-lauroyl-L-glutamate, sodium N-myristoyl-L-glutamate, sodium N-coconut-oil fatty acid-hardened tallow fatty acid-acyl-L-glutamate, potassium N-coconut-oil fatty acid-acyl-L-glutamate, and the like.

Moreover, flavors or fragrances may be mixed with the deodorant compositions. As a result, a strange odor characteristic to the substrate can be masked and further pleasant aroma can be also imparted.

The mixing amount of the flavor or fragrance varies depending on the polyphenol or a polymer thereof to be employed, sodium percarbonate and hydrogen peroxide to be employed, applied target of the deodorant composition, using method thereof and the like, but it is desirable to set it to generally from 0.001 times to 500 times by weight based on the deodorant composition.

The flavor to be used in the invention includes synthetic aroma chemicals such as esters, alcohols, aldehydes, ketones, acetals, phenols, ethers, lactones, furans, hydrocarbons, and acids, flavor materials of natural origin, and the like.

Examples of the esters in the synthetic aroma chemicals to be used as the above flavor preferably include acrylate esters (methyl, ethyl, etc.), acetoacetate esters (methyl, ethyl, etc.), anisate esters (methyl, ethyl, etc.), benzoate esters (allyl, isoamyl, ethyl, geranyl, linalyl, phenylethyl, hexyl, cis-3-hexenyl, benzyl, methyl, etc.), anthranilate esters (cinnamyl, cis-3-hexenyl, methyl, ethyl, linalyl, isobutyl, etc.), N-methylanthranilate esters (methyl, ethyl, etc.), isovalerate ester (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyrate esters (isoamyl, geranyl, citronellyl, terpenyl, cinnamyl, octyl, neryl, phenylethyl, phenylpropyl, phenoxyethyl, butyl, propyl, isopropyl, hexyl, benzyl, methyl, ethyl, linalyl, rhodinyl, etc.), undecylenate esters (allyl, isoamyl, butyl, ethyl, methyl, etc.), octanoate esters (allyl, isoamyl, ethyl, octyl, hexyl, butyl, methyl, linalyl, etc.), octenoate esters (methyl, ethyl, etc.), octynecarboxylate esters (methyl, ethyl, etc.), caproate esters (allyl, amyl, isoamyl, methyl, ethyl, isobutyl, propyl, hexyl, cis-3-hexenyl, trans-2-hexenyl, linalyl, geranyl, cyclohexyl, etc.), hexenoate esters (methyl, ethyl, etc.), valerate esters (amyl, isopropyl, isobutyl, ethyl, cis-3-hexenyl, trans-2-hexenyl, cinnamyl, phenylethyl, methyl, etc.), formate esters (anisyl, isoamyl, isopropyl, ethyl, octyl, geranyl, citronellyl, cinnamyl, cyclohexyl, terpinyl, phenylethyl, butyl, propyl, hexyl, cis-3-hexenyl, benzyl, linalyl, rhodinyl, etc.), crotonate esters (isobutyl, ethyl, cyclohexyl, etc.), cinnamate esters (allyl, ethyl, methyl, isopropyl, propyl, 3-phenylpropyl, benzyl, cyclohexyl, methyl, etc.), succinate esters (monomenthyl, diethyl, dimethyl, etc.), acetate esters (anisyl, amyl, α-amylcinnamyl, isoamyl, isobutyl, isopropyl, isopulegyl, isobornyl, isoeugenyl, eugenyl, 2-ethylbutyl, ethyl, 3-octyl, carvyl, dihydrocarvyl, p-cresyl, o-cresyl, geranyl, α- or β-santalyl, cyclohexyl, cycloneryl, dihydrocuminyl, dimethylbenzylcarbinyl, cinnamyl, styrallyl, decyl, dodecyl, terpinyl, guanyl, neryl, nonyl, phenylethyl, phenylpropyl, butyl, furfuryl, propyl, hexyl, cis-3-hexenyl, trans-2-hexenyl, cis-3-nonenyl, cis-6-nonenyl, cis-3, cis-6-nonadienyl, 3-methyl-2-butenyl, menthyl, heptyl, benzyl, bornyl, myrcenyl, dihydromyrcenyl, myrtenyl, methyl, 2-methylbutyl, menthyl, linalyl, rhodinyl, etc.), salicylate esters (allyl, isoamyl, phenyl, phenylethyl, benzyl, ethyl, methyl, etc.), cyclohexylalkanoate esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearate esters (ethyl, propyl, butyl, etc.), sebacate esters (diethyl, dimethyl, etc.), decanoate esters (isoamyl, ethyl, butyl, methyl, etc.), dodecanoate esters (isoamyl, ethyl, butyl, etc.), lactate esters (isoamyl, ethyl, butyl, etc.), nonanoate esters (ethyl, phenylethyl, methyl, etc.), nonenoate esters (allyl, ethyl, methyl, etc.), hydroxyhexanoate esters (ethyl, methyl, etc.), phenylacetate esters (isoamyl, isobutyl, ethyl, geranyl, citronellyl, cis-3-hexenyl, methyl, etc.), phenoxyacetate esters (allyl, ethyl, methyl, etc.), furancarboxylate esters (ethyl furancarboxylate, methyl furancarboxylate, hexyl furancarboxylate, isobutyl furanpropionate, etc.), propionate esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linalyl, terpinyl, α-methylpropionyl, β-methylpropionyl, etc.), heptanoate esters (allyl, ethyl, octyl, propyl, methyl, etc.), heptynecarboxylate esters (allyl, ethyl, propyl, methyl, etc.), myristate esters (isopropyl, ethyl, methyl, etc.), phenylglycidate esters (ethyl phenylglycidate, ethyl 3-methylphenylglycidate, ethyl p-methyl-β-phenylglycidate, etc.), 2-methylbutyrate esters (methyl, ethyl, octyl, phenylethyl, butyl, hexyl, benzyl, etc.), 3-methylbutyrate esters (methyl, ethyl, etc.), butyrate esters (anisyl, amyl, allyl, isoamyl, methyl, ethyl, propyl, octyl, guanyl, linalyl, geranyl, cyclohexyl, citronellyl, cinnamyl, neryl, terpenyl, phenylpropyl, β-phenylethyl, butyl, hexyl, cis-3-hexenyl, trans-2-hexenyl, benzyl, rhodinyl, etc.), hydroxybutyrate esters (3-hydroxybuyrate methyl, ethyl, or menthyl, etc.), and the like.

Examples of the alcohols to be used as the flavors in the invention preferably include aliphatic alcohols (isoamyl alcohol, isopulegol, 2-ethylhexanol, 1-octanol, 3-octanol, 1-octen-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.), terpene alcohols (carveol, borneol, isoborneol, carveol, piperitol, geraniol, α- or β-santalol, citronellol, 4-thujanol, terpineol, 4-terpineol, nerol, myrcenol, myrtenol, menthol, dihydromyrcenol, tetrahydromyrcenol, nerolidol, hydroxycitronerol, farnesol, perilla alcohol, rhodinol, linalool, etc.), aromatic alcohols (anise alcohol, α-amylcinnamic alcohol, isopropylbenzylcarbinol, carvacrol, cuminic alcohol, dimethylbenzylcarbinol, cinnamic alcohol, phenylallyl alcohol, phenylethylcarbinol, phenylethyl alcohol, 3-phenylpropyl alcohol, benzyl alcohol, etc.), and the like.

Examples of the aldehydes to be used as the flavors in the invention preferably include aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.), aromatic aldehydes (anisaldehyde, α-amylcinnamic aldehyde), α-methylcinnamic aldehyde, cyclamen aldehyde, p-isopropylphenylacetaldehyde, ethylvanillin, cuminaldehyde, salicylaldehyde, cinnamic aldehyde, o-, m- or p-tolylaldehyde, vanillin, piperonal, phenylacetaldehyde, heliotropin, benzaldehyde, 4-methyl-2-phenyl-2-pentenal, p-methoxycinnamic aldehyde, p-methoxybenzaldehyde, etc.), terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myrac aldehyde, vemaldehyde, safranal, etc.), and the like.

Preferred examples of the ketones to be used as the flavors in the invention preferably include cyclic ketones (menthone, isomenthone, carvone, dihydrocarvone, pulegone, piperitone, 1-acetyl-3,3-dimethyl-1-cyclohexene, cis-jasmone, α-, β- or γ-irone, ethylmaltol, cyclotene, dihydronootkatone, 3,4-dimethyl-1,2-cyclopentadione, sotolone, α-, β- γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.), aromatic ketones (acetonaphthone, acetophenone, anisylideneacetone, raspberry ketone, p-methylacetophenone, anisylacetone, p-methoxyacetophenone, etc.), linear ketones (diacetyl, 2-nonanone, diacetyl, 2-heptanone, 2,3-heptanedione, 2-pentanone, methyl amyl ketone, methyl nonyl ketone, β-methyl naphthyl ketone, methylheptanone, 3-heptanone, 4-heptanone, 3-octanone, 2,3-hexanedione, 2-undecanone, dimethyloctenone, 6-methyl-5-heptyn-3-one, etc.), and the like.

Preferred examples of the acetals to be used as the flavors in the invention include acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethyl propyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, ambersage (manufactured by Givaudan), ethyl acetoacetate ethylene glycol acetal, 2-phenylpropanal dimethyl acetal, and the like.

Preferred examples of the phenols to be used as the flavors in the invention include eugenol, isoeugenol, 2-methoxy-4-vinylphenol, thymol, carvacrol, guaiacol, chavicol, and the like.

Preferred examples of the ethers to be used as the flavors in the invention include anethole, 1,4-cineole, 1,8-cineole, dibenzyl ether, linalool oxide, limonene oxide, nerol oxide, rose oxide, methylisoeugenol, methyl chavicol, isoamyl phenylethyl ether, β-naphthyl methyl ether, phenyl propyl ether, p-cresyl methyl ether, vanillyl butyl ether, α-terpinyl methyl ether, citronellyl ethyl ether, geranyl ethyl ether, rose-furan, theaspirane, decyl methyl ether, methyl phenylmethyl ether, and the like.

Preferred examples of the lactones to be used as the flavors in the invention include γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyllactone, 5-hydroxy-8-undecenenoic acid δ-lactone, jasmine lactone, menthalactone, dihydrocoumarin, octahydrocoumarin, 6-methylcoumarin, and the like.

Preferred examples of the furans to be used as the flavors in the invention include furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, menthofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dimethyl-3-hydroxy-2(5H)-furanone (sotolone), 2-ethyl-4-hydroxy-5-methyl-3(2H)-furanone (homofuranol), 5-ethyl-3-hydroxy-4-methyl-2(5H)furanone (homosotolone), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, furfuryl acetate, and the like.

Preferred examples of the hydrocarbons to be used as the flavors in the invention include α-bisabolene, β-caryophyllene, p-cymene, terpinene, terpinolene, cadinene, farnesene, limonene, ocimene, myrcene, α- or β-pinene, 1,3,5-undecatriene, valencene, and the like.

Moreover, preferred examples of the acids to be used as the flavors in the invention include octanoic acid, nonanoic acid, decanoic acid, 2-decenoic acid, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, cyclohexanecarboxylic acid, and the like.

Furthermore, examples of the flavor materials of natural origin to be used as the flavors include anise, orange, lemon, lime, mandarin, petit grain, bergamot, lemon balm, grapefruit, elemi, olibanum, lemon grass, neroli, marjoram, angelica root, star anise, basil, bay, calamus, chamomile, caraway, cardamom, cassia, cinnamon, peppermint, spearmint, mint, penny royal, pepper, perilla, cypress, oregano, cascarilla, ginger, parsley, pine needle, sage, hyssop, tea tree, mustard, horseradish, clarisage, clove, cognac, coriander, estragon, eucalyptus, fennel, guaiac wood, dill, cajuput, worm seed, pimento, juniper, fenugreek, garlic, laurel, mace, mil, nutmeg, spruce, geranium, citronella, lavender, lavandin, palmarosa, rose, rosemary, sandalwood, oak moth, cider wood, vetiver, linaloe, bois de rose, patchouli, labdanum, cumin, thyme, ylang-ylang, birth, capsicum, celery, tolu balsam, djenne, inmortel, benzoin, jasmine, cassia, tuberose, mignonette, marigold, mimosa, opopanax, orris, vanilla, licorice, and the like. The flavor components contained in these flavor materials of natural origin can be also used.

The fragrance to be used in the invention includes hydrocarbons, alcohols, phenols, aldehydes and/or acetals, ketones and/or ketals, ethers, synthetic musks, acids, lactones, esters, halogen-containing compounds, fragrance materials of natural origin, and the like.

The hydrocarbons to be used as fragrances in the invention are not particularly limited as far as they are volatile organic compounds composed of carbon and hydrogen. Examples thereof include aliphatic hydrocarbons, alicyclic hydrocarbons, terpene hydrocarbons, aromatic hydrocarbons, and the like. Preferred examples include 1,3,5-undecatriene, p-cymene, α-pinene, α-phellandrene, β-caryophyllene, β-pinene, Δ-carene, allo-ocimene, ocimene, dihydromyrcene, dipentene, sclarene, cedrene, terpinene, terpinolene, valencene, bisabolene, farnesene, myrcene, limonene, longifolene, adamantane, isolongifolene, camphene, guaiene, diphenyl, diphenylmethane, biphenyl, 3,7-dimethyl-1,3,6-octatriene, 4-isopropyl-1-methyl-2-propenylbenzene, 7-methyl-3-methylene-1,6-octadiene, p-ethylstyrene, α-p-dimethylstyrene, isoprene, undecatriene, undecane, octadecadiene, octadecane, octadecene, octane, octene, cumene, sabinene, cyclohexane, cyclohexene, cyclopentadiene, dicyclopentadiene, styrene, decalin, decane, tetradecane, tetralin, dodecane, tridecane, tridecene, naphthalene, nonane, nonene, norbornane, norbornene, hexadecane, hexane, heptadecadiene, heptadecane, heptadecene, heptane, and pentadecane. Further preferred examples include 1,3,5-undecatriene, p-cymene, α-pinene, α-phellandrene, β-caryophyllene, β-pinene, Δ-carene, allo-ocimene, ocimene, dihydromyrcene, dipentene, sclarene, cedrene, terpinene, terpinolene, valencene, bisabolene, farnesene, myrcene, limonene, longifolene, adamantane, isolongifolene, and camphene.

The alcohols to be used as fragrances in the invention are not particularly limited as far as they are volatile organic compounds having a hydroxyl group. Examples thereof include aliphatic alcohols, alicyclic alcohols, terpene alcohols, aromatic alcohols, and the like. Preferred examples include 10-undecenol, 1-octen-3-ol, 2,6-nonadienol, 2-tert-butylcyclohexanol, 2-ethylhexanol, 2-heptanol, 3,5,5-trimethylhexanol, 3-octanol, 3-phenylpropyl alcohol, L-menthol, n-decyl alcohol, α-dimethylbenzyl alcohol, p-tert-butylcyclohexanol, p-methyldimethylbenzylcarbinol, α,3,3-trimethyl-2-norbornanemethanol, α-n-amylcinnamic alcohol, α-fenchyl alcohol, β-phenylethyl alcohol, anise alcohol, amber core, ambrinol, isononyl alcohol, isophytol, isopulegol, isoborneol, ethyllinalool, octanol, carveol, geraniol, santalol, cis-3-hexen-1-ol, cis-6-nonenol, citronellol, dihydro-α-terpineol, dihydrocitronellol, dihydromyrcenol, dihydrolinalool, dimethylphenylethylcarbinol, dimethylbenzylcarbinol, cinnamic alcohol, styrally alcohol, cedrol, terpineol, terpinen-4-ol, Timberol, tetrahydrogeraniol, tetrahydromyrcenol, tetrahydromugol, tetrahydrolinalool, nerol, nerolidol, nonanol, nonyl alcohol, nopol, hydrotropyl alcohol, Bacdanol, patchouli alcohol, farnesol, phytol, phenylethylmethylethylcarbinol, phenoxyethyl alcohol, furfuryl alcohol, vetivenol, perilla alcohol, benzyl alcohol, mayol, myrcenol, myrtenol, lavandulol, linalool, 1-(2,2,6-trimethylcyclohexanyl)-hexan-3-ol, 1,1-dimethyl-3-phenylpropanol, 1-decanol, 1-dodecanol, 1-nonen-3-ol, 1-heptanol, 1-penten-3-ol, 2,2-dimethyl-3-phenylpropanol, 2,4-dimethyl-3-cyclohexene-1-methanol, 2,4-dimethylbenzyl alcohol, 2,4-hexadienol, 2,5,5-trimethyloctahydro-2-naphthol, 2,6-dimethylheptan-2-ol, 2-isobutyl-4-hydroxy-4-methyltetrahydropyran, 2-undecanol, 2-octanol, 2-nonanol, 2-phenylpropyl alcohol, 2-methyl-3-buten-2-ol, 2-methyl-4-(2,2,3-trimethyl-3-cyclopentenyl)-2-butenol, 2-methyl-4-(2,2,3-trimethyl-3-cyclopentenyl)-butanol, 2-methyloctanol, 2-methyldecanol, 2-methoxy-2-phenylethyl alcohol, 3,3-dimethyl-Δ2,β-norbornane-2-ethanol, 3,4,5,6,6-pentamethyl-2-heptanol, 3,6-dimethyloctan-3-ol, 3,7-dimethyl-1-octanol, 3,7-dimethyl-7-methoxyoctan-2-ol, 3-thujanol, 3-dodecanol, 3-heptanol, 3-methyl-1-phenyl-3-pentanol, 3-methyl-2-buten-1-ol, 3-methyl-5-(2,2,3-trimethyl-3-cyclopentenyl)pentan-2-ol, 3-methyl-5-phenylpentanol, 3-methylpentanol, 4-isopropylcyclohexanol, 4-thujanol, 4-methyl-3-decen-5-ol, 5-methyl-2-phenyl-2-hexanol, 6,8-dimethyl-2-nonanol, 9-decenol, 9-decen-1-ol, E.G. monobutyl ether, sec-undecylic alcohol, sec-octyl alcohol, sec-nonyl alcohol, α,α,p-trimethylphenylethyl alcohol, α,α-dimethylphenylethyl alcohol, α-isobutylphenylethyl alcohol, α-bisabolol, α-propylphenylethyl alcohol, β,γ-hexenol, β-caryophyllene alcohol, γ-4-dimethyl-3-cyclohexene-1-propanol, allo-ocimenol, Ambestol, isocamphylcyclohexanol, isocyclogeraniol, isodihydrolavandulol, isobutylbenzylcarbinol, undecanol, ethylene glycol, ethylene glycol monoethyl ether, ethylene glycol monobutyl ether, ethylene glycol monopropyl ether, ethylene glycol monomethyl ether, ocimenol, Camekol DH, cumin alcohol, geranyllinalool, sabinene hydrate, diethylene glycol, diethylene glycol monoethyl ether, diethylene glycol monobutyl ether, diethylene glycol monopropyl ether, diethylene glycol monomethyl ether, cyclohexylethyl alcohol, cyclomethylene citronellol, cis-4-hexen-1-ol, cis-p-isopropylcyclohexylmethanol, dihydrocarveol, dipropylene glycol, dipropylene glycol monoethyl ether, dipropylene glycol monobutyl ether, dipropylene glycol monopropyl ether, dipropylene glycol monomethyl ether, dimethyloctanol, dimethylvinylcarbinol, sclareol, decahydro-β-naphthol, tetrahydroallo-ocimenol, trans-2-octanol, trans-2-hexenol, trans-3-hexen-1-ol, neopentyl glycol, hydrocinnamic alcohol, vanillyl alcohol, pinocarveol, butane-1,3-diol, butane-1,3-diol monoethyl ether, butane-1,3-diol monobutyl ether, butane-1,3-diol monopropyl ether, butane-1,3-diol monomethyl ether, butane-2,3-diol, butane-2,3-diol monoethyl ether, butane-2,3-diol monobutyl ether, butane-2,3-diol monopropyl ether, butane-2,3-diol monomethyl ether, butylene glycol, propylene glycol, propylene glycol monoethyl ether, propylene glycol monobutyl ether, propylene glycol monopropyl ether, propylene glycol monomethyl ether, hexamethylene glycol, hexylene glycol, pentamethylene glycol, muguet alcohol, methyl β-phenylethyl alcohol, and methyl sandeflor. Further preferred examples thereof include 10-undecenol, 1-octen-3-ol, 2,6-nonadienol, 2-tert-butylcyclohexanol, 2-ethylhexanol, 2-heptanol, 3,5,5-trimethylhexanol, 3-octanol, 3-phenylpropyl alcohol, L-menthol, n-decyl alcohol, α-dimethylbenzyl alcohol, p-tert-butylcyclohexanol, p-methyldimethylbenzylcarbinol, α,3,3-trimethyl-2-norbornanemethanol, α-n-amylcinnamic alcohol, α-fenchyl alcohol, β-phenylethyl alcohol, anise alcohol, amber core, ambrinol, isononyl alcohol, isophytol, isopulegol, isoborneol, ethyllinalool, octanol, carveol, geraniol, santalol, cis-3-hexen-1-ol, cis-6-nonenol, citronellol, dihydro-α-terpineol, dihydrocitronellol, dihydromyrcenol, dihydrolinalool, dimethylphenylethylcarbinol, dimethylbenzylcarbinol, cinnamic alcohol, styrally alcohol, cedrol, terpineol, terpinen-4-ol, Timberol, tetrahydrogeraniol, tetrahydromyrcenol, tetrahydromugol, tetrahydrolinalool, nerol, nerolidol, nonanol, nonyl alcohol, nopol, hydrotropyl alcohol, Bacdanol, patchouli alcohol, farnesol, phytol, phenylethylmethylethylcarbinol, phenoxyethyl alcohol, furfuryl alcohol, vetivenol, perillaalcohol, benzyl alcohol, myol, myrcenol, myrtenol, lavandulol, and linalool.

The phenols to be used as fragrances in the invention are not particularly limited as far as they are organic compounds which are phenolic compounds or derivatives thereof and have aroma or pleasant odor, and examples thereof include monovalent, divalent, or trivalent phenolic compounds, polyphenols, or ether derivatives thereof. Preferred examples thereof include p-cresol, isoeugenol, estragol, eugenol, hinokithiol, benzylisoeugenol, benzyleugenol, methylisoeugenol, methyleugenol, 2-methoxynaphthalene, 2,6-dimethoxyphenol, 4-ethylguaiacol, 4-methylguaiacol, 5-propenylguaetol, β-naphthol isobutyl ether, p-allylphenol, p-ethylphenol, isosafrol, ethylisoeugenol, catechol dimethyl ether, carvacrol, guaiacol, creosol, safrol, dihydroeugenol, thymol, chavicol, hydroquinone dimethyl ether, vanitrope, bromelia, methoxybenzene, resorcinol dimethyl ether, and shogaol.

The aldehydes or acetals to be used as fragrances in the invention are not particularly limited as far as they are volatile organic compounds having an aldehyde group or an acetal group in the molecule. Examples thereof include aliphatic aldehydes or acetals, terpene aldehydes or acetals, aromatic aldehydes or acetals, and the like. Preferred examples include 10-undecenal, 2,4-dimethyl-4,4-a,5,9b-tetrahydroindeno[1,2-d]-1,3-dioxine, 2,4-decadienal, 2,6-nonadienal, 2-butyl-4,4,6-trimethyl-1,3-dioxane, 2-hexyl-5-methyl-1,3-dioxolane, 2-methylundecanal, 2-methylundecanal dimethyl acetal, 3-ethyl-2,4-dioxaspiro[5.5]undec-8-ene, 3-ethyl-8(9),11-dimethyl-2,4-dioxaspiro[5.5]undec-8-ene, 3-propylbicyclo[2.2.1]-hept-5-ene-2-carboxyaldehyde, 4-isopropyl-5,5-dimethyl-1,3-dioxane, 4-heptenal, 5-methyl-5-propyl-2-(1-methylbutyl)-1,3-dioxane, o-methoxycinnamic aldehyde, o-methoxybenzaldehyde, p-tolylaldehyde, α-n-hexylcinnamic aldehyde, α-amylcinnamic aldehyde, acetaldehyde, acetaldehyde ethyl linalyl acetal, acetaldehyde diethyl acetal, anisaldehyde, aldehyde C-10, aldehyde C-11, aldehyde C-12, aldehyde C-6, aldehyde C-6 DEA, aldehyde C-6 DMA, aldehyde C-6 PG acetal, aldehyde C-8, aldehyde C-8 DEA, aldehyde C-8 DMA, aldehyde C-9, aldehyde C-9 DEA, aldehyde C-9 DMA, isocyclocitral, ethylvanillin, canthoxal, cucumber aldehyde, cumin aldehyde, geranial, cyclamen aldehyde, cis-6-nonenal, citral, citronellal, citronellyloxyacetaldehyde, cinensal, Dupical, trans-2-hexenal, trans-2-hexenal diethyl acetal, Triplal, neral, hydrotropaldehyde, vanillin, hydroxycitronellal, phenylacetaldehyde, phenylacetaldehyde P.G. acetal, phenylacetaldehyde dimethyl acetal, furfural, Floralozone, heliotropin, helional, perillaldehyde, bergamal, beltaldehyde, bemaldehyde, benzaldehyde, homo-myrac aldehyde, myrac aldehyde, melonal, lilal, lilial, 2,4,6-triisopropyl-1,3,5-trioxane, 2,4-undecadienal, 2,4-octadienal, 2,4-dioxa-3-methyl-7,10-methanospiro[5.5]-undecane, 2,4-dodecadienal, 2,4-nonadienal, 2,4-hexadienal, 2,4-heptadienal, 2,5,6-trimethyl-4-heptenal, 2,6,10-trimethyl-5,9-undecadienal, 2-methyl-3-(4-methylphenyl)-propanal, 2-methyl-4-(2,6,6-trimethyl-2-cyclohexenyl)-3-butenal, 2-methylbutanal, 3-phenylpropionic aldehyde, 3-phenylpropionic aldehyde dimethyl acetal, 3-methyl-5-phenylvaleraldehyde, 4-(2,2,6-trimethyl-2(1)-cyclohexene)-2-methylbutanal, 4-(4-methyl-3-cyclohexen-1-ylidene)-pentanal, 4-methyl-2-phenyl-2-pentenal, 5-(hydroxymethyl)-2-furfural, 5,9-dimethyl-4,9-decadienal, 5-methylfurfural, n-valeraldehyde, p-tert-butylhydrocinnamic aldehyde, p-isobutyl-α-methylhydrocinnamic aldehyde, p-isopropylhydrotropaldehyde, p-methylhydrotropaldehyde, p-methylphenylacetaldehyde, p-methylphenoxyacetaldehyde, p-methoxybenzaldehyde, α-n-amylcinnamic aldehyde diethyl acetal, α-amylcinnamic aldehyde dimethyl acetal, α-camphorenaldehyde, α-methylcinnamic aldehyde, β-methylhydrocinnamic aldehyde, γ-n-hexylcinnamic aldehyde, acetaldehyde ethyl isoeugenyl acetal, acetaldehyde ethyl cis-3-hexenyl acetal, acetaldehyde ethyl phenylethyl acetal, acetaldehyde ethyl hexyl acetal, acetaldehyde citronellyl ethyl acetal, acetaldehyde citronellyl methyl acetal, acetaldehyde phenylethyl n-propyl acetal, aldehyde C-13, aldehyde C-14, aldehyde C-5, aldehyde C-7, aldehyde C-7 DEA, aldehyde C-7 DMA, isovaleraldehyde, octahydro-4,7-methano-1H-indenecarboxyaldehyde, caryophyllenaldehyde, geranyloxyacetaldehyde, safranal, salicylaldehyde, cyclocitral, cis-3-hexenal, cis-3-hexenal diethyl acetal, cis-4-decenal, citral PG acetal, citral diethyl acetal, citral dimethyl acetal, citronellal EG acetal, dihydroindenyl-2,4-dioxane, dimethyloctanal, cinnamic aldehyde, decanal diethyl acetal, decanal dimethyl acetal, tetrahydrocitral, dodecanal dimethyl acetal, trans-2-undecenal, trans-2-decen-1-al, trans-2-dodecenal, trans-2-tridecenal, trans-2-nonenal, trans-2-heptenal, trans-2-pentenal, trans-4-decenal, trimethylundecenal, trimethyldecadienal, hydrotropaldehyde E.G. acetal, hydrotropaldehyde dimethyl acetal, vanillin P.G. acetal, paraldehyde, hydroxycitronellal diethyl acetal, phenylacetaldehyde 2,3-butylene glycol acetal, phenylacetaldehyde 2,4-dihydroxy-4-methylpentane acetal, phenylacetaldehyde diisobutyl acetal, phenoxyacetaldehyde, furfurylacrolein, heptanal E.G. acetal, heliotropin diethyl acetal, heliotropin dimethyl acetal, benzaldehyde PG acetal, benzaldehyde glyceryl acetal, benzaldehyde diethyl acetal, benzaldehyde dimethyl acetal, formaldehyde cyclododecyl ethyl acetal, methyldecanal, methylnonylacetaldehyde dimethyl acetal, methylvanillin, methoxydicyclopentadienecarboxyaldehyde, and methoxycitronellal. Further preferred examples include 10-undecenal, 2,4-dimethyl-4,4-a,5,9b-tetrahydroindeno[1,2-d]-1,3-dioxine, 2,4-decadienal, 2,6-nonadienal, 2-butyl-4,4,6-trimethyl-1,3-dioxane, 2-hexyl-5-methyl-1,3-dioxolane, 2-methylundecanal, 2-methylundecanal dimethyl acetal, 3-ethyl-2,4-dioxaspiro[5.5]undec-8-ene, 3-ethyl-8(9),11-dimethyl-2,4-dioxaspiro[5.5]undec-8-ene, 3-propylbicyclo[2.2.1]-hept-5-ene-2-carboxyaldehyde, 4-isopropyl-5,5-dimethyl-1,3-dioxane, 4-heptenal, 5-methyl-5-propyl-2-(1-methylbutyl)-1,3-dioxane, o-methoxycinnamic aldehyde, o-methoxybenzaldehyde, p-tolylaldehyde, α-n-hexylcinnamic aldehyde, α-amylcinnamic aldehyde, acetaldehyde, acetaldehyde ethyl linalyl acetal, acetaldehyde diethyl acetal, anisaldehyde, aldehyde C-10, aldehyde C-11, aldehyde C-12, aldehyde C-6, aldehyde C-6 DEA, aldehyde C-6 DMA, aldehyde C-6 PG acetal, aldehyde C-8, aldehyde C-8 DEA, aldehyde C-8 DMA, aldehyde C-9, aldehyde C-9 DEA, aldehyde C-9 DMA, isocyclocitral, ethylvanillin, kantokisal, cucumber aldehyde, cumin aldehyde, geranial, cyclamen aldehyde, cis-6-nonenal, citral, citronellal, citronellyloxyacetaldehyde, cinensal, Dupical, trans-2-hexenal, trans-2-hexenal diethyl acetal, Triplal, neral, hydrotropaldehyde, vanillin, hydroxycitronellal, phenylacetaldehyde, phenylacetaldehyde P.G. acetal, phenylacetaldehyde dimethyl acetal, furfural, Floralozone, heliotropin, helional, perillaldehyde, bergamal, beltaldehyde, bernaldehyde, benzaldehyde, homo-myrac aldehyde, myrac aldehyde, melonal, lilal, and lilial.

The ketones or ketals to be used as fragrances in the invention are not particularly limited as far as they are volatile organic compounds having a ketone group or a ketal group in the molecule include aliphatic ketones or ketals, terpene ketones or ketals, aromatic ketones or ketals, and the like. Preferred examples include 2-sec-butylcyclohexanone, 2-acetyl-3,3-dimethylnorbornane, 2-acetyl-5-methylfuran, 2-acetylfuran, 2-butyl-1,4-dioxaspiro[4,4]nonane, 2-hexylcyclopentanone, 3-hydroxy-4,5-dimethyl-2-(5H)-furanone, 5-ethyl-3-hydroxy-4-methyl-2[5H]-furanone, 6-methyl-3,5-heptadien-2-one, d-pulegone, L-carvone, o-tert-butylcyclohexanone, p-tert-butylcyclohexanone, p-methylacetophenone, p-methoxyacetophenone, α-dinascone, α-fenchone, β-methylnaphthylketone, acetylcedrene, acetophenone, anisylacetone, allyl α-ionone, ionone, iso-E-super, isojasmone, isodamascone, isolongifolanone, irone, ethyl isoamyl ketone, ethylmaltol, Cashmeran, carone, camphor, Koavone, cyclotene, cis-jasmone, dihydrocarvone, dihydrojasmone, dibenzyl ketone, cedorenone, sotolone, damascone, damascenone, trimofix O, nootkatone, furaneol, plicatone, florex, vertfix, verbenone, benzophenone, maltol, methylionone, methylcyclopentenolone, methylheptenone, menthone, raspberry ketone, 1-(4-methoxyphenyl)-1-penten-3-one, 1-(p-menthen-6-yl)-1-propanone, 1-acetyl-3,3-dimethyl-1-cyclohexene, 2-(1-cyclohexen-1-yl)cyclohexanone, 2,2,5,5-tetramethyl-4-isopropyl-1,3-dioxane, 2,2,5-trimethyl-5-pentylcyclopentanone, 2,3,5-trimethylcyclohexen-4-yl-1-methyl ketone, 2,3-hexadione, 2,3-heptanedione, 2,3-pentadione, 2,4-di-tert-butylcyclohexanone, 2,5,5-trimethyl-2-phenyl-1,3-dioxane, 2,6,10-trimethyl-1-acetyl-2,5,9-cyclododecatriene, 2,6,6-trimethyl-2-cyclohexene-1,4-dione, 2-n-butylidene-3,5,5(3,3,5)-trimethylcyclohexanone, 2-n-heptylcycloheptanone, 2′-acetonaphthone, 2-undecanone, 2-octanone, 2-cyclopentylcyclopentanone, 2-tridecanone, 2-nonanone, 2-hydroxy-6-isopropyl-3-methyl-2-cyclohexenone, 2-butanone, 2-heptanone, 2-heptylcyclopentanone, 2-pentanone, 2-pentyl-2-cyclopentenone, 2-pentylcyclopentanone, 3,3-dimethylcyclohexyl methyl ketone, 3,4-dimethyl-1,2-cyclopentadione, 3,4-hexadione, 3,5-dimethyl-1,2-cyclopentadione, 3-acetyl-2,5-dimethylfuran, 3-octanone, 3-nonanone, 3-hydroxymethyl-2-nonanone, 3-hexanone, 3-heptanone, 3-hepten-2-one, 3-methyl-4-phenyl-3-buten-2-one, 3-methyl-5-(2,2,3-trimethyl-3-cyclopentenyl)-3-penten-2-one, 3-methyl-5-propyl-2-cyclohexenone, 4-(4-hydroxy-3-methoxyphenyl)-2-butanone, 4-(4-methoxyphenyl)-3-buten-2-one, 4(5)-acetyl-7,7,9-(7,9,9)-trimethylbicyclo[4.3.0]nona-1-ene, 4,7-dihydro-2-(3-pentanyl)-1,3-dioxepine, 4,7-dihydro-2-isoamyl-2-methyl-1,3-dioxiepine, 4-tert-amylcyclohexanone, 4-oxoisophorone, 4-cyclohexenyl-4-methyl-2-pentanone, 4-heptanone, 4-methyl-3-penten-2-one, 4-methyl-4-phenyl-2-pentanone, 4-methylene-3,5,6,6-tetramethyl-2-heptanone, 5-cyclohexadecen-1-one, 5-hydroxy-4-octanone, 5-phenyl-5-methyl-3-hexanone, 5-methyl-2,3-hexadione, 7-methyl-3,5-dihydro-2H-benzodioxepin-3-one, p-hydroxyphenylbutanone, p-methoxyphenylacetone, α-methylanisalacetone, acetylisovaleryl, acetyl caryophyllene, acetyldimethyltetrahydrobenzindane, acetoin, acetoketal, acetophenone neopentyl glycol acetal, acetone, atrinone, anisylidenacetone, amylcyclopentanone, ethyl acetoacetate E.G. ketal, ethyl acetoacetate propylene glycol acetal, oxocedrane, cryptone, geranylacetone, diacetyl, diacetone alcohol, diosphenol, cyclohexanone, cyclohexenone, cyclopentanone, cis-2-acetonyl-4-methyltetrahydropyran, dimethyloctenone, zingerol, cedranone, vitalide, piperitenone, piperitone, piperonylacetone, farnesylacetone, pseudoionone, butylideneacetone, furfural acetone, propiophenone, heliotropylacetone, verdoxane, benzylideneacetone, homofuraneol, mesityl oxide, methyl α-furyl ketone, methyl isopropyl ketone, methyliritone, methylcedorilone, and methyltetrahydrofuranone. Further preferred examples include 2-sec-butylcyclohexanone, 2-acetyl-3,3-dimethylnorbornane, 2-acetyl-5-methylfuran, 2-acetylfuran, 2-butyl-1,4-dioxaspiro[4,4]nonane, 2-hexylcyclopentanone, 3-hydroxy-4,5-dimethyl-2-(5H)-furanone, 5-ethyl-3-hydroxy-4-methyl-2[5H]-furanone, 6-methyl-3,5-heptadien-2-one, d-pulegone, L-carvone, o-tert-butylcyclohexanone, p-tert-butylcyclohexanone, p-methylacetophenone, p-methoxyacetophenone, α-dinascone, α-fenchone, P-methylnaphthylketone, acetylcedrene, acetophenone, anisylacetone, allyl α-ionone, ionone, iso E super, isojasmone, isodamascone, isolongifolanone, irone, ethyl isoamyl ketone, ethylmaltol, Cashmeran, carone, camphor, Koavone, cyclotene, cis-jasmone, dihydrocarvone, dihydrojasmone, dibenzyl ketone, cedorenone, sotolone, damascone, damascenone, trimofix O, nootkatone, furaneol, plicatone, florex, vertfix, verbenone, benzophenone, maltol, methylionone, methylcyclopentenolone, methylheptenone, menthone, raspberry ketone.

The ethers to be used as fragrances in the invention are not particularly limited as far as they are volatile organic compounds having an ether group in the molecule. Examples thereof include aliphatic ethers, terpene ethers, aromatic ethers, and the like. Preferred examples include 1,4-cineol, 1,8-cineol, p-cresyl methyl ether, β-caryophyllene oxide, β-naphthyl isobutyl ether, β-naphthyl ethyl ether, β-naphthyl methyl ether, anethole, ambroxane, isoamyl phenylethyl ether, isobornyl methyl ether, grisalva, cyclamber, diphenyl oxide, cedrambar, cedryl methyl ether, teaspyran, nerol oxide, phenylethyl methyl ether, madrox, linalool oxide, limetol, Rhubofix, rhouboflor, rose oxide, rose furan, 13-oxabicyclo[10.3.0]pentadecane, 1-methylcyclododecyl methyl ether, 2,2,6-trimethyl-6-vinyltetrahydrofuran, 2,2-dimethyl-5-(1-methyl-1-propenyl)-tetrahydropyran, 2-ethylidene-6-isopropoxybicyclo[2.2.1]heptane, 2-oxaspiro[4.7]dodecane, 2-butyl-4,6-dimethyldihydropyran, 2-methyl-2-butenylphenyl ethyl ether, 3,3,5-trimethylcyclohexyl ethyl ether, 3-oxabicyclo[10.3.0]-pentadec-6-ene, 4-allylanisole, 5-isopropenyl-2-methyl-2-vinyltetrahydrofuran, 8,9-epoxycedrene, n-decyl vinyl ether, tert-butylhydroquinone dimethyl ether, α-cedrene epoxide, α-terpinyl methyl ether, allyl phenylethyl ether, isoamyl benzyl ether, isolongifolene epoxide, ethyl o-methoxybenzyl ether, ocimene epoxide, geranyl ethyl ether, cyclodecenyl methyl ether, cyclohexyl ethyl ether, cyclohexyl phenylethyl ether, citroxide, citroneryl ethyl ether, dibenzyl ether, juniparome, cedrol methyl ether, decyl methyl ether, tricyclodecenyl methyl ether, trimethylcyclododecatriene epoxide, methylphenyl ethyl ether, methyl hexyl ether, methyl benzyl ether, limonene oxide, 1,2-dimethoxybenzene, 1,3-dimethoxybenzene, 1,4-dimethoxy-2-tert-butylbenzene, ethylene glycol diethyl ether, ethylene glycol dibutyl ether, ethylene glycol dipropyl ether, ethylene glycol dimethyl ether, diethyl ether, diethylene glycol diethyl ether, diethylene glycol dibutyl ether, diethylene glycol dipropyl ether, diethylene glycol dimethyl ether, dimethyl ether, tetrahydrofuran, propylene glycol diethyl ether, and propylene glycol dimethyl ether. Further preferred examples thereof include 1,4-cineol, 1,8-cineol, p-cresyl methyl ether, β-caryophyllene oxide, β-naphthyl isobutyl ether, β-naphthyl ethyl ether, β-naphthyl methyl ether, anethole, ambroxane, isoamyl phenylethyl ether, isobornyl methyl ether, grisalva, cyclamber, diphenyl oxide, cedrambar, cedryl methyl ether, teaspyran, nerol oxide, phenylethyl methyl ether, madrox, linalool oxide, limetol, Rhubofix, rhouboflor, rose oxide, and rose furan.

The synthetic musks to be used as fragrances in the invention are not particularly limited as far as they are organic compounds having musk odor or musk-like odor and examples thereof include 10-oxahexadecanolide, 11-oxahexadecanolide, 12-oxahexadecanolide, ambrettolide, Ambreton, exaltolide, exaltone, Galaxolide, cyclohexadecanolide, cyclopentadecanolide, cyclopentadecanone, civetone, cervolide, celestolide, Tonalide, fantolide, pentalide, formylethyltetramethyltetralin, muscone, versalide, and the like.

The acids to be used as fragrances in the invention are not particularly limited as far as they are organic compounds having a carboxyl group in the molecule and examples thereof include phenylacetic acid, 2-ethylbutyric acid, 2-ethylhexanoic acid, 2-decenoic acid, 2-hexenoic acid, 2-methyl-2-pentenoic acid, 2-methylbutyric acid, 2-methylheptanoic acid, 4-pentenoic acid, 4-methylpentanoic acid, undecanoic acid, undecylenic acid, octanoic acid, oleic acid, geranic acid, cinnamic acid, stearic acid, tiglic acid, decanoic acid, dodecanoic acid, tridecanoic acid, nonanoic acid, hydrocinnamic acid, pyruvic acid, propionic acid, hexanoic acid, heptanoic acid, myristic acid, lactic acid, linolic acid, linoleic acid, levulinic acid, oxalic acid, glutalic acid, citric acid, succinic acid, tartaric acid, terephthalic acid, vanillic acid, valine, phytic acid, fumaric acid, benzoic acid, malic acid, maleic acid, malonic acid, and the like.

The lactones to be used as fragrances in the invention are not particularly limited as far as they are organic compounds having a lactone group in the molecule and examples thereof include aliphatic lactones, terpene lactones, aromatic lactones, and the like. Preferred examples thereof include 6-methylcoumarin, α-angelica lactone, γ-n-butyrolactone, γ-undecalactone, γ-octalactone, γ-decalactone, γ-nonalactone, γ-valerolactone, γ-hexylactone, γ-heptalactone, δ-2-decenolactone, δ-undecalactone, δ-octalactone, δ-decalactone, δ-tetradecalactone, δ-dodecalactone, δ-tridecalactone, δ-nonalactone, δ-hexylactone, ε-decalactone, ε-dodecalactone, aldehyde C-14 (peach), aldehyde C-18 (coconut), whisky lactone, dihydrojasmone lactone, jasmine lactone, jasmolactone, methyl γ-decalactone, menthalactone, 4,6,6(4,4,6)-trimethyltetrahydropyran-2-one, 7-decene-1,4-lactone, octahydrocoumarin, dihydrocoumarin, dodecalactone, 3-n-butylidenephthalide, 3-n-butylphthalide, 3-propylidenephthalide, and 3-propylphthalide.

The esters to be used as fragrances in the invention are not particularly limited as far as they are volatile organic compounds having an ester group in the molecule and examples thereof include aliphatic esters, terpene esters, aromatic esters, and the like. Preferred examples thereof include 1-ethynylcyclohexyl acetate, 1-octen-3-yl acetate, 2-ethylhexyl acetate, 2-phenoxyethyl isobutyrate, 2-phenoxyethyl propionate, 3,5,5-trimethylhexyl acetate, 3,7-dimethyloctanyl acetate, 3-phenylpropyl acetate, 9-decen-1-yl acetate, L-menthyl acetate, L-menthyl propionate, o-tert-butylcyclohexyl acetate, p-tert-butylcyclohexyl acetate, p-cresyl acetate, p-cresyl isobutyrate, p-cresyl phenylacetate, acetylisoeugenol, acetyleugenol, anisyl acetate, aphermate, amyl acetate, amyl caprylate, amyl caproate, amyl salicylate, amyl valerate, amyl butyrate, amyl formate, allyl 2-ethylbutyrate, allylamyl glycolate, allyl isovalerate, allyl octanoate, allyl caprylate, allyl caproate, allyl cyclohexylacetate, allyl cyclohexyloxyacetate, allyl cyclohexylbutyrate, allyl cyclohexylpropionate, allyl cinnamate, allyl phenoxyacetate, allyl butyrate, allyl heptanoate, allyl benzoate, aldehyde C-16 (strawberry), aldehyde C-19 (pineapple), aldehyde C-20 (raspberry), isoamyl acetate, isoamyl angelate, isoamyl isovalerate, isoamyl isobutyrate, isoamyl undecylenate, isoamyl octanoate, isoamyl salicylate, isoamyl cinnamate, isoamyl decanoate, isoamyl dodecanoate, isoamyl butyrate, isoamyl propionate, isoamyl hexanoate, isoamyl heptyne carbonate, isoamyl benzoate, isoamyl formate, isoamyl levulinate, isoeugenyl phenylacetate, isodihydrolavandulyl acetate, isobutyl acetate, isobutyl isovalerate, isobutyl isobutyrate, isobutyl salicylate, isobutyl cinnamate, isobutyl valerate, isobutyl phenylacetate, isobutyl butyrate, isobutyl propionate, isobutyl hexanoate, isobutyl benzoate, isopulegyl acetate, isopropyl acetate, isopropyl isovalerate, isopropyl isobutyrate, isopropyl cinnamate, isopropyl decanoate, isopropyl phenylacetate, isopropyl butyrate, isopropyl hexanoate, isopropyl benzoate, isopropyl myristate, isobornyl acetate, isobornyl propionate, Winter Green, ethyl 2-tert-butylcyclohexyl carbonate, ethyl 2-ethylhexanoate, ethyl 2-octenoate, ethyl 2-decenoate, ethyl 2-furoate, ethyl 2-hexylacetoacetate, ethyl 2-benzylacetoacetate, ethyl 2-methylvalerate, ethyl 2-methylbutyrate, ethyl 3,5,5-trimethylhexanoate, ethyl 3-hydroxybutyrate, ethyl 3-hydroxyhexanoate, ethyl 3-hydroxy-3-phenylpropionate, ethyl 3-phenylglycidate, ethyl 3-phenylpropionate, ethyl o-methoxybenzoate, ethyl p-anisate, ethyl acetate, ethyl acetoacetate, ethyl isovalerate, ethyl isobutyrate, ethyl octyne carbonate, ethyl oleate, ethyl caprinate, ethyl caprylate, ethyl caproate, ethyl crotonate, ethyl geranate, ethyl safranate, ethyl salicylate, ethyl cyclogeraniate, ethyl cinnamate, ethyl valerate, ethyl phenylacetate, ethyl butyrate, ethyl propionate, ethyl heptanoate, ethyl heptyne carbonate, ethyl pelargonate, ethyl benzoate, ethyl formate, ethyl myristate, ethyl methyl p-tolylglycidate, ethyl methyl phenylglycidate, ethyl laurate, ethyl lactate, ethyl linalylacetate, ethyl levulinate, ethylene dodecanedioate, ethylene brassylate, eugenyl phenylacetate, octyl acetate, octyl isovalerate, octyl isobutyrate, octyl octanoate, octyl butyrate, octyl heptanoate, octyl formate, ocimenyl acetate, caryophyllen acetate, caryophyllen formate, calyxol, carvyl acetate, guaiac acetate, cuminyl acetate, geranyl acetate, geranyl isovalerate, geranyl isobutylate, geranyl tiglate, geranyl phenylacetate, geranyl butyrate, geranyl propionate, geranyl hexanoate, geranyl benzoate, geranyl formate, coniferin, santaryl acetate, diethyl adipate, diethyl succinate, diethyl sebacate, diethyl tartrate, diethyl phthalate, diethyl malonate, cyclohexyl acetate, cyclohexyl isovalerate, cyclohexylethyl acetate, cyclohexyl crotonate, cyclohexyl butyrate, cis-3-hexenyl 2-methylbutyrate, cis-3-hexenyl acetate, cis-3-hexenyl angelate, cis-3-hexenyl isovalerate, cis-3-hexenyl isobutyrate, cis-3-hexenyl caproate, cis-3-hexenyl salicylate, cis-3-hexenyl tiglate, cis-3-hexenyl valerate, cis-3-hexenyl phenylacetate, cis-3-hexenyl butyrate, cis-3-hexenyl propionate, cis-3-hexenyl benzoate, cis-3-hexenyl formate, cis-3-hexenyl lactate, citryl acetate, citronellyl acetate, citronellyl isovalerate, citronellyl isobutyrate, citronellyl tiglate, citronellyl phenylacetate, citronellyl butylate, citronellyl propionate, citronellyl hexanoate, citronellyl formate, dihydrocarvyl acetate, dihydrocuminyl acetate, dihydroterpinyl acetate, dihydromyrcenyl acetate, dimethyl succinate, dimethylphenylethylcarbinyl acetate, dimethyl phthalate, dimethylbenzylcarbinyl acetate, dimethylbenzylcarbinyl isobutyrate, dimethylbenzylcarbinyl butyrate, dimethylbenzylcarbinyl propionate, jasmal, cinnamyl acetate, cinnamyl isovalerate, cinnamyl isobutyrate, cinnamyl cinnamate, cinnamyl tiglate, cinnamyl butyrate, cinnamyl propionate, cinnamyl benzoate, cinnamyl formate, styrallyl acetate, styrallyl isobutyrate, styrallyl propionate, cedryl acetate, cedryl formate, terpinyl acetate, terpinyl isovalerate, terpinyl isobutyrate, terpinyl butyrate, terpinyl propionate, terpinyl formate, decahydro-β-naphthyl formate, decyl acetate, tetrahydrofurfuryl butyrate, tetrahydrogeranyl acetate, tetrahydrofurfuryl acetate, tetrahydromugyl acetate, tetrahydrolinalyl acetate, dodecyl acetate, trans-2-hexenyl acetate, trans-2-hexenyl butyrate, trans-2-hexenyl propionate, trans-2-hexenyl hexanoate, trans-decahydro-β-naphthyl acetate, trans-decahydro-β-naphthyl isobutyrate, triacetin, triethyl citrate, tricyclodecyl acetate, tricyclodecenyl acetate, tricyclodecenyl isobutyrate, tricyclodecenyl propionate, neryl acetate, neryl isobutyrate, neryl butyrate, neryl propionate, neryl formate, nonyl acetate, nopyl acetate, hydrotropic acetate, phenylethyl 2-methylbutyrate, phenylethyl acetate, phenylethyl angelate, phenylethyl isovalerate, phenylethyl isobutyrate, phenylethyl caprylate, phenylethyl salicylate, phenylethyl cinnamate, phenylethyl tiglate, phenylethyl nonanoate, phenylethyl valerate, phenylethyl pivalate, phenylethyl phenylacetate, phenylethyl butyrate, phenylethyl propionate, phenylethyl benzoate, phenylethyl formate, phenylethyl methacrylate, phenylethylmethylethylcarbinyl acetate, phenyl salicylate, fenchyl acetate, butyl acetate, butyl angelate, butyl isovalerate, butyl isobutyrate, butyl octanoate, butyl salicylate, butyl decanoate, butyl dodecanoate, butyl valerate, butyl phenylacetate, butyl butyryllactate, butyl butyrate, butyl propionate, butyl hexanoate, butyl levulinate, furfuryl acetate, prenyl acetate, prenyl angelate, prenyl benzoate, propyl acetate, propyl isovalerate, propyl isobutyrate, propyl octanoate, propyl cinnamate, propyl trans-2, cis-4-decadienoate, propyl phenylacetate, propyl butyrate, propyl propionate, propyl hexanoate, propyl heptanoate, propyl benzoate, propyl formate, hexyl 2-methylbutyrate, hexyl acetate, hexyl isovalerate, hexyl isobutyrate, hexyl octanoate, hexyl salicylate, hexyl tiglate, hexyl phenylacetate, hexyl butyrate, hexyl propionate, hexyl hexanoate, hexyl benzoate, hexyl formate, veticol acetate, vetiveryl acetate, heptyl acetate, heptyl octanoate, heptyl butyrate, heptyl hexanoate, heliotropyl acetate, benzyl 2-methylbutyrate, benzyl acetate, benzyl isovalerate, benzyl isobutyrate, benzyl caprylate, benzyl salicyalte, benzyl cinnamate, benzyl tiglate, benzyl dodecanoate, benzyl valerate, benzyl phenylacetate, benzyl butyrate, benzyl propionate, benzyl hexanoate, benzyl benzoate, benzyl formate, pentyl salicylate, myraldyl acetate, myrcenyl acetate, myrtenyl acetate, methyl 1-methyl-3-cyclohexenecarboxylate, methyl 2-nonenoate, methyl 2-furoate, methyl 2-methylbutyrate, methyl 3-nonenoate, methyl 9-undecenoate, methyl o-methoxybenzoate, methyl acetate, methyl atrarate, methyl anisate, methyl angelate, methyl isovalerate, methyl isobutyrate, methyl isohexanoate, methyl octanoate, methyl octyne carbonate, methyl oleate, methyl caprinate, methyl caprylate, methyl caproate, methyl geranate, methyl salicylate, methyl cyclooctyl carbonate, methyl cyclogeranate, methyl cyclopentylideneacetate, methyl dihydrojasmonate, methyl jasmonate, methyl cinnamate, methyl decanoate, methyl decyne carbonate, methyl tetradecanoate, methyl dodecanoate, methyl trans-2-hexenoate, methyl trans-3-hexenoate, methyl nonanoate, methyl hydroxyhexanoate, methyl valerate, methyl phenylacetate, methyl phenylglycidate, methyl butyrate, methyl heptanoate, methyl heptyne carbonate, methyl pelargonate, methyl benzoate, methyl myristate, methyl laurate, methyl lactate, lavandulyl acetate, linalyl acetate, linalyl isovalerate, linalyl isobutyrate, linalyl octanoate, linalyl cinnamate, linalyl butyrate, linalyl propionate, linalyl hexanoate, linalyl benzoate, linalyl formate, rosamusk, rosephenone, rhodinyl acetate, rhodinyl isobutyrate, rhodinyl phenylacetate, rhodinyl butyrate, rhodinyl propionate, rhodinyl formate, 1,3-dimethyl-3-butenyl isobutyrate, 1-acetoxy-2-sec-butyl-1-vinylcyclohexane, 1-cyclohex-1-ene isopropylacetate, 2,4-dimethyl-3-cyclohexylmethyl acetate, 2,4-hexadienyl isobutyrate, 2-methyl-2-methylpentyl valerate, 2-methylbutyl acetate, 2-methylbutyl isovalerate, 3-octyl acetate, 3-phenylpropyl isovalerate, 3-phenylpropyl isobutyrate, 3-phenylpropyl propionate, 3-methylpentyl angelate, 4-methylbenzyl acetate, 5-methyl-3-butyltetrahydropyran-4-yl acetate, 6,10-dimethyl-5,9-undecatrien-2-yl acetate, 9-decen-1-yl propionate, E.G. diacetate, E.G. monobutyl ether acetate, L-carvyl propionate, L-perillyl acetate, L-bornyl propionate, L-menthyl isovalerate, L-menthyl phenylacetate, P.G. dibutyrate, P.G. dipropionate, p-cresyl caprylate, p-cresyl salicylate, α-amylcinnamyl acetate, acetylvanillin, anisyl propionate, anisyl formate, isobutyl 2-furanpropionate, isobutyl angelate, isobutyl crotonate, ethyl acrylate, ethyl citronellyl oxalate, ethyl stearate, ethyl tiglate, ethyl decadienoate, ethyl dehydrocyclogeranate, ethyl dodecanoate, ethyl trans-2-hexanoate, ethyl trans-3-hexanoate, ethyl nonanoate, ethyl palmitate, ethyl valerate, ethyl pyruvate, eugenyl formate, oxyoctalin formate, nerolidyl acetate, nonanediol-1,3-diacetate, phenyl glycol diacetate, pseudolinalyl acetate, butyl 10-undecenoate, butyl stearate, butyl formate, butyl lactate, furfuryl valerate, and propyl 2-furanacrylate. Further preferred examples include 1-ethynylcyclohexyl acetate, 1-octen-3-yl acetate, 2-ethylhexyl acetate, 2-phenoxyethyl isobutyrate, 2-phenoxyethyl propionate, 3,5,5-trimethylhexyl acetate, 3,7-dimethyloctanyl acetate, 3-phenylpropyl acetate, 9-decen-1-yl acetate, L-menthyl acetate, L-menthyl propionate, o-tert-butylcyclohexyl acetate, p-tert-butylcyclohexyl acetate, p-cresyl acetate, p-cresyl isobutyrate, p-cresyl phenylacetate, acetylisoeugenol, acetyleugenol, anisyl acetate, aphermate, amyl acetate, amyl caprylate, amyl caproate, amyl salicylate, amyl valerate, amyl butyrate, amyl formate, allyl 2-ethylbutyrate, allylamyl glycolate, allyl isovalerate, allyl octanoate, allyl caprylate, allyl caproate, allyl cyclohexylacetate, allyl cyclohexyloxyacetate, allyl cyclohexylbutyrate, allyl cyclohexylpropionate, allyl cinnamate, allyl phenoxyacetate, allyl butyrate, allyl heptanoate, allyl benzoate, aldehyde C-16 (strawberry), aldehyde C-19 (pineapple), aldehyde C-20 (raspberry), isoamyl acetate, isoamyl angelate, isoamyl isovalerate, isoamyl isobutyrate, isoamyl undecylenate, isoamyl octanoate, isoamyl salicylate, isoamyl cinnamate, isoamyl decanoate, isoamyl dodecanoate, isoamyl butyrate, isoamyl propionate, isoamyl hexanoate, isoamyl heptyne carbonate, isoamyl benzoate, isoamyl formate, isoamyl levulinate, isoeugenyl phenylacetate, isodihydrolavandulyl acetate, isobutyl acetate, isobutyl isovalerate, isobutyl isobutyrate, isobutyl salicylate, isobutyl cinnamate, isobutyl valerate, isobutyl phenylacetate, isobutyl butyrate, isobutyl propionate, isobutyl hexanoate, isobutyl benzoate, isopulegyl acetate, isopropyl acetate, isopropyl isovalerate, isopropyl isobutyrate, isopropyl cinnamate, isopropyl decanoate, isopropyl phenylacetate, isopropyl butyrate, isopropyl hexanoate, isopropyl benzoate, isopropyl myristate, isobornyl acetate, isobornyl propionate, ethyl 2-tert-butylcyclohexyl carbonate, ethyl 2-ethylhexanoate, ethyl 2-octenoate, ethyl 2-decenoate, ethyl 2-furoate, ethyl 2-hexylacetoacetate, ethyl 2-benzylacetoacetate, ethyl 2-methylvalerate, ethyl 2-methylbutyrate, ethyl 3,5,5-trimethylhexanoate, ethyl 3-hydroxybutyrate, ethyl 3-hydroxyhexanoate, ethyl 3-hydroxy-3-phenylpropionate, ethyl 3-phenylglycidate, ethyl 3-phenylpropionate, ethyl o-methoxybenzoate, ethyl p-anisate, ethyl acetate, ethyl acetoacetate, ethyl isovalerate, ethyl isobutyrate, ethyl octyne carbonate, ethyl oleate, ethyl caprinate, ethyl caprylate, ethyl caproate, ethyl crotonate, ethyl geranate, ethyl safranate, ethyl salicylate, ethyl cyclogeraniate, ethyl cinnamate, ethyl valerate, ethyl phenylacetate, ethyl butyrate, ethyl propionate, ethyl heptanoate, ethyl heptyne carbonate, ethyl pelargonate, ethyl benzoate, ethyl formate, ethyl myristate, ethyl methyl p-tolylglycidate, ethyl methyl phenylglycidate, ethyl laurate, ethyl lactate, ethyl linalylacetate, ethyl levulinate, ethylene dodecanedioate, ethylene brassylate, eugenyl phenylacetate, octyl acetate, octyl isovalerate, octyl isobutyrate, octyl octanoate, octyl butyrate, octyl heptanoate, octyl formate, ocimenyl acetate, caryophyllen acetate, caryophyllen formate, carvyl acetate, guaiac acetate, cuminyl acetate, geranyl acetate, geranyl isovalerate, geranyl isobutylate, geranyl tiglate, geranyl phenylacetate, geranyl butyrate, geranyl propionate, geranyl hexanoate, geranyl benzoate, geranyl formate, coniferin, santaryl acetate, diethyl adipate, diethyl succinate, diethyl sebacate, diethyl tartrate, diethyl phthalate, diethyl malonate, cyclohexyl acetate, cyclohexyl isovalerate, cyclohexylethyl acetate, cyclohexyl crotonate, cyclohexyl butyrate, cis-3-hexenyl 2-methylbutyrate, cis-3-hexenyl acetate, cis-3-hexenyl angelate, cis-3-hexenyl isovalerate, cis-3-hexenyl isobutyrate, cis-3-hexenyl caproate, cis-3-hexenyl salicylate, cis-3-hexenyl tiglate, cis-3-hexenyl valerate, cis-3-hexenyl phenylacetate, cis-3-hexenyl butyrate, cis-3-hexenyl propionate, cis-3-hexenyl benzoate, cis-3-hexenyl formate, cis-3-hexenyl lactate, citryl acetate, citronellyl acetate, citronellyl isovalerate, citronellyl isobutyrate, citronellyl tiglate, citronellyl phenylacetate, citronellyl butylate, citronellyl propionate, citronellyl hexanoate, citronellyl formate, dihydrocarvyl acetate, dihydrocuminyl acetate, dihydroterpinyl acetate, dihydromyrcenyl acetate, dimethyl succinate, dimethylphenylethylcarbinyl acetate, dimethyl phthalate, dimethylbenzylcarbinyl acetate, dimethylbenzylcarbinyl isobutyrate, dimethylbenzylcarbinyl butyrate, dimethylbenzylcarbinyl propionate, jasmal, cinnamyl acetate, cinnamyl isovalerate, cinnamyl isobutyrate, cinnamyl cinnamate, cinnamyl tiglate, cinnamyl butyrate, cinnamyl propionate, cinnamyl benzoate, cinnamyl formate, styrallyl acetate, styrallyl isobutyrate, styrallyl propionate, cedryl acetate, cedryl formate, terpinyl acetate, terpinyl isovalerate, terpinyl isobutyrate, terpinyl butyrate, terpinyl propionate, terpinyl formate, decahydro-β-naphthyl formate, decyl acetate, tetrahydrofurfuryl butyrate, tetrahydrogeranyl acetate, tetrahydrofurfuryl acetate, tetrahydromugyl acetate, tetrahydrolinalyl acetate, dodecyl acetate, trans-2-hexenyl acetate, trans-2-hexenyl butyrate, trans-2-hexenyl propionate, trans-2-hexenyl hexanoate, trans-decahydro-β-naphthyl acetate, trans-decahydro-β-naphthyl isobutyrate, triacetin, triethyl citrate, tricyclodecyl acetate, tricyclodecenyl acetate, tricyclodecenyl isobutyrate, tricyclodecenyl propionate, neryl acetate, neryl isobutyrate, neryl butyrate, neryl propionate, neryl formate, nonyl acetate, nopyl acetate, hydrotropic acetate, phenylethyl 2-methylbutyrate, phenylethyl acetate, phenylethyl angelate, phenylethyl isovalerate, phenylethyl isobutyrate, phenylethyl caprylate, phenylethyl salicylate, phenylethyl cinnamate, phenylethyl tiglate, phenylethyl nonanoate, phenylethyl valerate, phenylethyl pivalate, phenylethyl phenylacetate, phenylethyl butyrate, phenylethyl propionate, phenylethyl benzoate, phenylethyl formate, phenylethyl methacrylate, phenylethylmethylethylcarbinyl acetate, phenyl salicylate, fenchyl acetate, butyl acetate, butyl angelate, butyl isovalerate, butyl isobutyrate, butyl octanoate, butyl salicylate, butyl decanoate, butyl dodecanoate, butyl valerate, butyl phenylacetate, butyl butyryllactate, butyl butyrate, butyl propionate, butyl hexanoate, butyl levulinate, furfuryl acetate, prenyl acetate, prenyl angelate, prenyl benzoate, propyl acetate, propyl isovalerate, propyl isobutyrate, propyl octanoate, propyl cinnamate, propyl trans-2, cis-4-decadienoate, propyl phenylacetate, propyl butyrate, propyl propionate, propyl hexanoate, propyl heptanoate, propyl benzoate, propyl formate, hexyl 2-methylbutyrate, hexyl acetate, hexyl isovalerate, hexyl isobutyrate, hexyl octanoate, hexyl salicylate, hexyl tiglate, hexyl phenylacetate, hexyl butyrate, hexyl propionate, hexyl hexanoate, hexyl benzoate, hexyl formate, veticol acetate, vetiveryl acetate, heptyl acetate, heptyl octanoate, heptyl butyrate, heptyl hexanoate, heliotropyl acetate, benzyl 2-methylbutyrate, benzyl acetate, benzyl isovalerate, benzyl isobutyrate, benzyl caprylate, benzyl salicyalte, benzyl cinnamate, benzyl tiglate, benzyl dodecanoate, benzyl valerate, benzyl phenylacetate, benzyl butyrate, benzyl propionate, benzyl hexanoate, benzyl benzoate, benzyl formate, pentyl salicylate, myraldyl acetate, myrcenyl acetate, myrtenyl acetate, methyl 1-methyl-3-cyclohexenecarboxylate, methyl 2-nonenoate, methyl 2-furoate, methyl 2-methylbutyrate, methyl 3-nonenoate, methyl 9-undecenoate, methyl o-methoxybenzoate, methyl acetate, methyl atrarate, methyl anisate, methyl angelate, methyl isovalerate, methyl isobutyrate, methyl isohexanoate, methyl octanoate, methyl octyne carbonate, methyl oleate, methyl caprinate, methyl caprylate, methyl caproate, methyl geranate, methyl salicylate, methyl cyclooctyl carbonate, methyl cyclogeranate, methyl cyclopentylideneacetate, methyl dihydrojasmonate, methyl jasmonate, methyl cinnamate, methyl decanoate, methyl decyne carbonate, methyl tetradecanoate, methyl dodecanoate, methyl trans-2-hexenoate, methyl trans-3-hexenoate, methyl nonanoate, methyl hydroxyhexanoate, methyl valerate, methyl phenylacetate, methyl phenylglycidate, methyl butyrate, methyl heptanoate, methyl heptyne carbonate, methyl pelargonate, methyl benzoate, methyl myristate, methyl laurate, methyl lactate, lavandulyl acetate, linalyl acetate, linalyl isovalerate, linalyl isobutyrate, linalyl octanoate, linalyl cinnamate, linalyl butyrate, linalyl propionate, linalyl hexanoate, linalyl benzoate, linalyl formate, rosamusk, rosephenone, rhodinyl acetate, rhodinyl isobutyrate, rhodinyl phenylacetate, rhodinyl butyrate, rhodinyl propionate, and rhodinyl formate.

The halogen-containing compounds to be used as fragrances in the invention are not particularly limited as far as they are harogenide having aroma or pleasant odor, and examples thereof include p-dichlorobenzene and bromostyrol.

The fragrance materials of natural origin to be used as fragrances in the invention are not particularly limited and examples thereof include almond oil, anise oil, abies-far oil, amyris oil, angelica oil, ambergris tincture, amber sage, ambret seed oil, ylang-ylang oil, incense oil, winter green oil, elemi oil, oak moth absolute, oak moth essence, oak moth oil, opoponax oil, orris absolute, orange oil, orange flower absolute, cascarilla oil, castoreum resinoid, cassia china oil, cassia absolute, cassia oil, cananga java oil, chamomile oil blue, chamomile oil, calamus oil, cardamom oil, galbanum oil, caraway oil, guaiac wood oil, guaiac oil, cumin oil, clove boulbon oil, clove oil, costus oil, copaiba balsam, copaiba oil, coriander oil, cypress oil, sandalwood oil, cystlabdanum oil, ceder wood oil, citronella oil, civet absolute, jasmine absolute, juniper berry oil, camphor oil, jonquille absolute, ginger oil, ginger grass oil, cinnamon Ceylon oil, sweet fennel oil, styrax oil, spike lavender oil, spearmint oil, sage oil, sage clary oil, geranium oil, geranium grass oil, geranium Bourbon oil, celery oil, thyme oil, tarragon oil, tangerine oil, tuberose absolute, tolu balsam oil, Diptheryx odorata oil, nutmeg oil, narcissus absolute, neroli bigarado oil, verbena oil, violet leaves absolute, pine oil, basil oil, parsley seed oil, patchouli oil, vanilla oil, vanilla resinoid, hyssop oil, bitter almond oil, bitter fennel oil, Hinoki oil, hiba oil, pimento berry oil, hyacinth absolute, petit-grain oil, buchu oil, bay oil, petit-grain grass oil, petit-grain Paraguay oil, petit-grain bergamot oil, petit-grain mandarin, petit-grain lemon oil, vetiver oil Java, vetiver Bourbon, pennyroyal oil, pepper oil, peppermint oil, Peru balsam, Peru balsam oil, bergamot oil, benzoin oil, benzoin resinoid, bois de rose oil, ho oil, horwood oil, marjoram oil, mandarin oil, mimosa absolute, mil oil, musk tonkin tincture, mace oil, merrissa oil, eucalyptus oil, lime oil, lavandine oil, labdanum oil, lavender oil, roux oil, lemon oil, lemongrass oil, rose do mai, rose Bulgar oil, rosemary oil, Roman chamomile oil, laurel oil, and lovage oil. These natural materials can be used in various forms such as essential oils, resinoids, balsams, absolutes, concretes, and tinctures.

The following show chemical names of the trade names and general names in the above materials.

Dupical (Quest); 4-(tricyclo[5.2.1.02,6]decylidene-8)butanal.

Jasmal; 3-pentyltetrahydropyran-4-yl acetate.

Aphermate (IFF); α,3,3-trimethylcyclohexanemethyl formate.

Floralozon (IFF); p-ethyl-α,α-dimethylhydrocinnamaldehyde.

Cyclogalbanate (Dragoco); allyl cyclohexyloxyacetate.

Estragole; methyl chavicol.

Rhubofix (Firmenich); an isomeric mixture of spiro[1,4-methanonaphthlene-2(1H), 2′-oxirane],3,4,4a,5,8,8a,-hexahydro-3′,7-dimethyl (1) and spiro[1,4-methanonaphthalene-(2H), 2′-oxiran],3,4,4a,5,8,8a-hexahydro-3′,6-dimethyl (2).

Triplal (IFF); dimethyl tetrahydrobenzaldehyde.

Koavone (IFF); 4-methylene-3,5,6,6-tetramethyl-2-heptanone.

Limetol; 2,2,6-trimethyl-6-vinyl tetrahydropyran.

Ambroxan (Henkel).

Damascone; α-damascone, β-damascone, γ-damascone, δ-damascone.

Damascenone; α-damascenone, β-damascenone, γ-damascenone.

Ionone; α-ionone, β-ionone, γ-ionone.

Methylionone; α-n-methylionone, β-n-methylionone, γ-n-methylionone, α-iso-methylionone, β-iso-methylionone, γ-iso-methylionone.

Sandal: bacdanol (IFF); 2-ethyl-4-(2,2,3-trimethyl-3-cyclopenten-1-yl)-2-buten-1-ol,

Brahamanol (Dragoco); 2-methyl-4-(2,2,3-trimethyl-3-cyclopenten-1-yl)butanol,

Madranol (Dragoco); P-2,2,3-tetramethyl-3-cyclopentenyl-2-butenol,

Sandalore (Givaudan); 3-methyl-5-(2,2,3-trimethylcyclopent-3-en-1-yl)-pentan-2-ol, 3,3-dimethyl-5-(2,2,3-trimethylcyclopenten-1-yl)-pent-4-en-2-ol,

Methyl sandeflor (TPC), Sandeol (MS), and the like.

Musk: Cashmeran (IFF), Galaxolide (IFF), Tonalid (PFW), phantolid, versalide, exaltolide, exaltone, oxalide, 12-oxahexadecanolide, ethylenebrassylate, celestolide (IFF), Traseolide (Quest), ethylenedodecanedioate, 5-cyclohexadecen-1-one, and the like.

Iso-E-Super (IFF); 7-acetyl-1,2,3,4,5,6,7,8-octahydro-1,1,6,7-tetramethyl-naphthalene.

Timberol (Dragoco); 1-(2,2,6-trimethylcyclohexan-1-yl)-hexan-3-ol.

Irone; α-irone, β-irone, γ-irone.

α-Dynascone (Firmenich); 1-(5,5-dimethylcyclohexen-1-yl)-4-penten-1-one.

Furthermore, in addition to the above flavors and fragrances, use can be made of flavors and fragrances described in “Nippon niokeru Shokuhin Koryo Kagoubutsu no Siyou Jittai Chosa” (2000^{th} Kosei Kagaku Kenkyu Hokokusho; Nihon Koryo Kogyokai, published on March, 2001), “Gosei koryo—Kgaku to Shohin Chisiki” (published on Mar. 6, 1996, written by Motoichi Indoh, The Chemical Daily Co., Ltd.), “Perfume and Flavor Chemicals (Aroma Chemicals) 1, 2” (Steffen Arctender (1969)).

These flavors and fragrances may be used solely or as mixtures of two or more of them.

As these substances, commercially available substances can be also used. Moreover, as a single substance, a synthetic substance or substance which purified from a natural material such as a plant may be used. The essential oils, resinoids, balsams, absolutes, concretes, tinctures, and the like can be also prepared by known methods.

As the stabilizer, there may be mentioned thickening polysaccharides, xanthan gum, pectin, carrageenan, gellan gum, locust gum, guar gum, alginic acid, cellulose, gum arabic, casein sodium and the like.

As the antifoaming agent, there may be mentioned silicone oil, a mineral oil-based antifoaming agent, a surfactant such as a fatty acid ester, a higher alcohol, a polyether and the like.

### <Malodor Component as Target>

As the malodor component to be deodorized by the use of the deodorant composition of the invention, illustratively, there may be mentioned sulfides (sulfur-containing compounds) such as dimethyl sulfide, dimethyl disulfide and dimethyl trisulfide. In addition, as the target to be deodorized, there may be mentioned, for example, malodor caused by a disease such as cancer, body odor, oral odor, smell of raw garbage, smell of tobacco, smell generated in toilet room, bathroom and the like, malodor generated from sewage, waste water and septage, smell of permanent preparation and the like. Illustratively, a malodor caused by breast cancer can be mentioned as the malodor caused by a disease such as cancer, and in “Identification of Malodorous Substance Generated from Affected Parts of Advanced Cancer (written in Japanese)” edited by Mika Shirasu and Kazunari Higashihara, AROMA RESEARCH, No. 40, Vol. 10/No. 4, 2009, P. 347”, there is a description that the malodorous substance of breast cancer is dimethyl trisulfide.

### <Using Form of Deodorant Composition>

Form of the deodorant composition of the invention can be optionally determined in response to the above-mentioned target to be deodorized and use (using purpose). As illustrative examples of said form, there may be mentioned a liquid form (an aqueous solvent extract and an extract thereof are included), a solid form such as a powder form, a granular form and a tablet form, a creamy form (a paste form and a gel form are included), a spray preparation form (a spray form) and the like.

The deodorant composition of the invention can achieve its desired deodorization effect when a malodor component is allowed to contact with the deodorant composition of the invention, and its embodiment of actual use is not limited at all with the proviso that this can contact with a malodor. For example, in carrying out deodorization of a limited space such as a kitchen, a refrigerator, a toilet room and an inner part of a room, it can be effected by arranging or spraying the deodorant of the invention in said limited space. In carrying out deodorization of a disease odor caused by a disease such as a cancer, for example, it can be effected by directly applying it to the affected part or covering its periphery therewith.

### EXAMPLES

The following describes the invention further in detail based on examples and comparative examples, but the invention is not limited to the following examples.

### <Deodorization Test 1>

### (Preparation of Polyphenol Polymer)

1,600 ml of 50 mM sodium carbonate solution was added to 80 g of dried leaves of green tea, followed by vigorously stirring at 30° C. for 1 hour (pH of the reaction solution was 8.7). The reaction liquid was filtered and then the filtrate was freeze-dried to obtain 251.8 g (64% based on dried leaves) of a polyphenol polymer.

### Examples 1 and 2, Reference Examples 1 to 3, Comparative Examples 1 and 2

Each of the deodorants having the compositions shown in Table 1 was collected in a 20 ml capacity HS vial and each of them was dissolved by adding 2 ml of distilled water. After adding 10 μl of dimethyl trisulfide (DMTS) 1% (v/v) ethanol solution as the malodor component thereto, the contents were stirred at room temperature for 2.5 hours after putting a cap on the HS vial. An SPME fiber was inserted into a head space part and allowed to carry out adsorption at room temperature for 0.5 hour.

Residual ratio of DMTS was calculated by comparing the peak area value of DMTS by GC-FID measurement with that of the control in which the deodorant composition was not used. The results are shown in Table 1.

TABLE 1

| | | | | | | Comparative | |
|---|---|---|---|---|---|---|---|
| | Examples | | Reference Examples | | | Examples | |
| Deodorant component | 1 | 2 | 1 | 2 | 3 | 1 | 2 |
| Polyphenol polymer | 10 mg | — | — | 10 mg | — | — | — |
| Gallic acid | — | 10 mg | — | — | 10 mg | — | — |
| Phyllostachys pubescens | — | — | — | — | — | 10 mg | — |
| extract (*1) | | | | | | | |
| Champignon extract (*2) | — | — | — | — | — | — | 10 mg |
| Sodium percarbonate | 100 mg | 100 mg | 100 mg | — | — | — | — |
| Residual ratio of DMTS | 13.0 | 7.2 | 32.3 | 94.2 | 93.6 | 94.2 | 100.0 |
| (%) | | | | | | | |

(*1) Phyllostachys pubescens extract: NEOBAMBUS-1000 ® (manufactured by SHIRAIMATSU PHARMACEUTICAL CO., LTD.)
(*2) Champignon extract: trade name Bio-M BX100FPD (manufactured by RICOM CORPORATION)

Based on the results of Table 1, it was able to eliminate dimethyl trisulfide to some degree by the sodium percarbonate alone (Reference Example 1) in comparison with Comparative Examples 1 and 2 which are conventional deodorants, but the deodorization effect of the dimethyl trisulfide by polyphenol alone (Reference Examples 2 and 3) was almost the same degree or only slightly improved. Contrary to this, in the Examples 1 and 2 in which sodium percarbonate and polyphenol polymer were used in combination, the deodorization effect for dimethyl trisulfide was significantly expressed and it was found that the effect is synergistically increased.

### <Deodorization Test 2>

A deodorization test on various sulfides as offensive smell components was carried out by the following method. In this connection, the polyphenol polymer as the deodorizing component in the deodorant composition, which was obtained in the above, was used.

Each of the deodorant compositions having the compositions shown in Table 2 was collected in a 20 ml capacity HS vial and each of them was dissolved by adding 2 ml of distilled water. After adding 10 μl of sulfide solution (1% (v/v) ethanol solution) or trimethylamine solution as the malodor component, shown in Table 2, thereto, the contents were stirred at room temperature after putting a cap on the HS vial. An SPME fiber was inserted into a head space part and allowed to carry out adsorption at room temperature for 0.5 hour.

The test was carried out by 4 systems, i.e. deodorant no addition system, polyphenol polymer addition system, sodium percarbonate addition system and polyphenol polymer and sodium percarbonate addition system, and the sulfide residual ratio in each system was evaluated by regarding GC Area of sulfides in the deodorant no addition system as 100%.

Since the reactivity and adsorption efficiency to SPME fiber differ depending on the kind of malodor component, the test was carried out by adjusting the adding amount of deodorant, reaction time and GC split ratio as shown in Table 2.

TABLE 2

| | | | Deodorant composition | | | | Sulfide |
|---|---|---|---|---|---|---|---|
| | | | Polyphenol | Sodium | Reaction | GC split | residual |
| | | Malodor tested | polymer | percarbonate | time (hr) | ratio | ratio (%) |
| A | 1 | Dimethyl sulfide | 10 mg | — | 0.5 | 1/5 | 97.5 |
| | 2 | | — | 10 mg | | | 43.2 |
| | 3 | | 10 mg | 10 mg | | | 24.2 |
| B | 1 | Dimethyl disulfide | 10 mg | — | 3.0 | 1/50 | 96.8 |
| | 2 | | — | 100 mg | | | 76.0 |
| | 3 | | 10 mg | 100 mg | | | 5.6 |
| C | 1 | Dimethyl trisulfide | 10 mg | | 3.0 | 1/50 | 94.2 |
| | 2 | | — | 100 mg | | | 32.3 |
| | 3 | | 10 mg | 100 mg | | | 13.0 |
| D | 1 | Diallyl sulfide | 10 mg | — | 3.0 | 1/50 | 83.2 |
| | 2 | | — | 50 mg | | | 44.1 |
| | 3 | | 10 mg | 50 mg | | | 10.4 |
| E | 1 | Diallyl disulfide | 10 mg | — | 3.0 | 1/50 | 94.4 |
| | 2 | | — | 100 mg | | | 100.0 |
| | 3 | | 10 mg | 100 mg | | | 43.2 |
| F | 1 | Diallyl trisulfide | 10 mg | — | 3.0 | 1/50 | 100.0 |
| | 2 | | — | 100 mg | | | 80.2 |
| | 3 | | 10 mg | 100 mg | | | 55.4 |
| G | 1 | Allylmethyl sulfide | 10 mg | — | 0.5 | 1/50 | 100.0 |
| | 2 | | — | 10 mg | | | 92.3 |
| | 3 | | 10 mg | 10 mg | | | 62.4 |
| H | 1 | Diisopropyl | 20 mg | — | 3.0 | 1/50 | 100.0 |
| | 2 | disulfide | — | 200 mg | | | 100.0 |
| | 3 | | 20 mg | 200 mg | | | 68.6 |

Based on the results of Table 2, synergistic effect by the combined use of polyphenol polymer and sodium percarbonate was found on all of the sulfides.

In taking note of the number of sulfur atoms of sulfides, high synergistic effect was found in both cases of dimethyl and diallyl in their form of disulfide. Based on this, it was found that the system regarding disulfide is the system from which the effect of the invention can be obtained most suitably, because it is markedly stable for sodium percarbonate and the effect of their combined use is markedly high.

On the other hand, the system regarding sulfide wherein the number of sulfur atoms is 1 has a tendency of showing low synergistic effect, but this is because sodium percarbonate is very effective, as a result, it shows a markedly deodorizing tendency. In addition, the system regarding trisulfide shows an intermediary property between the system regarding sulfide and the system regarding disulfide.

Regarding the alkyl group, it was found that its symmetric types have a tendency of easily causing deodorization in order of dimethyl, diallyl and diisopropyl.

### <Deodorization Test 3>

A deodorization test on various sulfides as malodor components was carried out by the following method. In this connection, the polyphenol polymer as the deodorizing component in the deodorant composition, which was obtained in the above, was used.

Each of the deodorant compositions having the compositions shown in Table 3 was collected in a 20 ml capacity HS vial and each of them was dissolved by adding 2 ml of distilled water. After adding 10 μl of sulfide solution (1% (v/v) ethanol solution) as the malodor component, shown in Table 3, thereto, the contents were stirred at room temperature for 3 hours after putting a cap on the HS vial. The cap was took off after the reaction and malodor in the vial was sniffed and its strength was evaluated.

The test was carried out by 4 systems, i.e. deodorant no addition system, polyphenol polymer addition system, sodium percarbonate addition system and polyphenol polymer and sodium percarbonate addition system. The evaluation was carried out by three special panels by regarding a case in which the strength was not different in comparison with that of the malodor in the deodorant no addition system as 4 points, a case in which the strength became slightly weak as 3 points, a case in which the strength became obviously weak as 2 points and a case in which the strength became sharply weak as 1 point.

The results are shown in Table 3. In this connection, the numerical value in the table is an evaluation obtained by synthesizing evaluations of the three special panels.

TABLE 3

| | | | Deodorant composition | | |
|---|---|---|---|---|---|
| | | | Polyphenol | Sodium | Strength of |
| | | Malodor tested | polymer | percarbonate | malodor |
| B | 1 | Dimethyl disulfide | 10 mg | — | 4 |
| | 2 | | — | 100 mg | 3 |
| | 3 | | 10 mg | 100 mg | 1 |
| E | 1 | Diallyl disulfide | 10 mg | — | 4 |
| | 2 | | — | 100 mg | 4 |
| | 3 | | 10 mg | 100 mg | 1 |
| F | 1 | Diallyl trisulfide | 10 mg | — | 4 |
| | 2 | | — | 100 mg | 3 |
| | 3 | | 10 mg | 100 mg | 1 |
| G | 1 | Allylmethyl sulfide | 10 mg | — | 4 |
| | 2 | | — | 10 mg | 4 |
| | 3 | | 10 mg | 10 mg | 2 |
| H | 1 | Diisopropyl | 20 mg | — | 4 |
| | 2 | disulfide | — | 200 mg | 4 |
| | 3 | | 20 mg | 200 mg | 2 |

Based on the results of Table 3, it was found that the synergistic effect by combined use of polyphenol polymer and sodium percarbonate was found on all of the sulfides. It was shown that the method of the invention can be applied broadly to sulfides.

### <Deodorization Test 4>

The above-mentioned polyphenol polymer and each of the oxidizing agents shown in Table 4 were collected in a 20 ml capacity HS vial and each of them was dissolved by adding 2 ml of distilled water. After adding 10 μl of dimethyl trisulfide (DMTS) 1% (v/v) ethanol solution as the malodor component thereto, the contents were stirred at room temperature for 3 hours after putting a cap on the HS vial. An SPME fiber was inserted into a head space part and allowed to carry out adsorption at room temperature for 0.5 hour.

Residual ratio of DMTS was calculated by comparing the peak area value of DMTS by GC-FID measurement with that of the control. In this connection, the GC split ratio was adjusted to 1/50. The results are shown in Table 4.

In addition, by measuring deodorization ratio of each test system (sulfide deodorization ratio when the no addition system was regarded as 1.00), synergetic property of the combined use effect of polyphenol polymer and each oxidizing agent was calculated by the following formula (I). The results are shown in Table 5.

Synergetic property=combined use÷(polyphenol+oxidizing agent) (1)

(* Judged synergistic effect present when synergetic property≧1)

TABLE 4

| | | | | | Dimethyl |
|---|---|---|---|---|---|
| | | | Deodorant composition | | trisulfide |
| | | | Polyphenol | Oxidizing | Residual |
| | | Oxidizing agent | polymer | agent | ratio (%) |
| J | 1 | Sodium percarbonate | 10 mg | — | 94.2 |
| | 2 | | — | 100 mg | 32.3 |
| | 3 | | 10 mg | 100 mg | 13.0 |
| K | 1 | Sodium perborate | 10 mg | — | 94.2 |
| | 2 | | — | 50 mg | 10.3 |
| | 3 | | 10 mg | 50 mg | 8.8 |
| L | 1 | Hydrogen peroxide | 10 mg | — | 94.2 |
| | 2 | 30% aqueous solution | — | 100 μl | 84.8 |
| | 3 | | 10 mg | 100 μl | 49.0 |
| M | 1 | Sodium hypochlorite | 10 mg | — | 94.2 |
| | 2 | | — | 1 μl | 32.3 |
| | 3 | | 10 mg | 1 μl | 99.5 |

TABLE 5

| | | Deodorization ratio | | | |
|---|---|---|---|---|---|
| | | | | Combined | |
| | | Poly- | | use of | |
| | | phenol | Oxidizing | polyphenol | |
| | | polymer | agent | polymer and | Synergetic |
| | Oxidizing agent | alone | alone | oxidizing agent | property |
| J | Sodium | 0.06 | 0.68 | 0.87 | 1.18 |
| | percarbonate: | | | | |
| | 100 mg | | | | |
| K | Sodium | 0.06 | 0.90 | 0.91 | 0.95 |
| | perborate: 50 mg | | | | |
| L | Hydrogen | 0.06 | 0.15 | 0.51 | 2.43 |
| | peroxide 30% | | | | |
| | aqueous solution: | | | | |
| | 100 μl | | | | |
| M | Sodium | 0.06 | 0.68 | 0.01 | 0.01 |
| | hypochlorite: 1 μl | | | | |

* For the sake of calculation, a case in which the deodorization ratio showed a negative value was corrected to the minimum value (0.01)

Based on the results of Table 4 and Table 5, similar to the case of sodium percarbonate, it was able to confirm evident synergistic effect also in the case of the use of its degradation product, i.e. hydrogen peroxide. On the other hand, it was found that sodium hypochlorite shows strong deodorization effect by its single use but the effect is inhibited when jointly used with polyphenol. In addition, since sodium perborate has such a strong effect by its single use, its synergistic effect was not able to be confirmed.

While the invention has been described in detail and with reference to specific embodiments thereof, it will be apparent to one skilled in the art that various changes and modifications can be made therein without departing from the spirit and scope thereof.

This application is based on Japanese Patent Application No. 2010-214196 filed on Sep. 24, 2010, and the entire subject matter of which is incorporated herein by reference.

### INDUSTRIAL APPLICABILITY

Since the deodorant composition for sulfides of the invention specifically acts upon malodors derived particularly from sulfides, it can be used in respective fields as a deodorant which deodorizes the malodor in the medical and care taking fields, the smell of domestic raw garbage, the smell of tobacco/cigarette, the smell generated in toilet room and bathroom, and the like.

1. A deodorant composition for sulfides, which comprises (A) a polyphenol or a polymer thereof and (B) sodium percarbonate or hydrogen peroxide.

2. The deodorant composition for sulfides according to claim 1, wherein the polymer of the polyphenol is a compound having a molecular weight of 10,000 or less which is obtained by allowing the polyphenol to undergo a reaction in an alkaline solvent.

3. The deodorant composition for sulfides according to claim 1, wherein a mixing ratio ((A):(B)) of the component (A) to the component (B) is from 1:0.1 to 1:1,000 in terms of mass ratio.

4. The deodorant composition for sulfides according to claim 1, wherein the component (A) is the polymer of the polyphenol.

5. The deodorant composition for sulfides according to claim 4, wherein the polymer of the polyphenol is a compound obtained by allowing a tea extract to undergo a reaction in an alkaline solvent.

6. The deodorant composition for sulfides according to claim 1, wherein the polyphenol is at least one species selected from the group consisting of a raw coffee bean extract, a tea extract and gallic acid.

7. The deodorant composition for sulfides according to claim 2, wherein a mixing ratio ((A):(B)) of the component (A) to the component (B) is from 1:0.1 to 1:1,000 in terms of mass ratio.

8. The deodorant composition for sulfides according to claim 2, wherein the component (A) is the polymer of the polyphenol.

9. The deodorant composition for sulfides according to claim 3, wherein the component (A) is the polymer of the polyphenol.

10. The deodorant composition for sulfides according to claim 7, wherein the component (A) is the polymer of the polyphenol.

11. The deodorant composition for sulfides according to claim 8, wherein the polymer of the polyphenol is a compound obtained by allowing a tea extract to undergo a reaction in an alkaline solvent.

12. The deodorant composition for sulfides according to claim 9, wherein the polymer of the polyphenol is a compound obtained by allowing a tea extract to undergo a reaction in an alkaline solvent.

13. The deodorant composition for sulfides according to claim 10, wherein the polymer of the polyphenol is a compound obtained by allowing a tea extract to undergo a reaction in an alkaline solvent.

14. The deodorant composition for sulfides according to claim 2, wherein the polyphenol is at least one species selected from the group consisting of a raw coffee bean extract, a tea extract and gallic acid.

15. The deodorant composition for sulfides according to claim 3, wherein the polyphenol is at least one species selected from the group consisting of a raw coffee bean extract, a tea extract and gallic acid.

16. The deodorant composition for sulfides according to claim 4, wherein the polyphenol is at least one species selected from the group consisting of a raw coffee bean extract, a tea extract and gallic acid.

17. The deodorant composition for sulfides according to claim 7, wherein the polyphenol is at least one species selected from the group consisting of a raw coffee bean extract, a tea extract and gallic acid.

18. The deodorant composition for sulfides according to claim 8, wherein the polyphenol is at least one species selected from the group consisting of a raw coffee bean extract, a tea extract and gallic acid.

19. The deodorant composition for sulfides according to claim 9, wherein the polyphenol is at least one species selected from the group consisting of a raw coffee bean extract, a tea extract and gallic acid.

20. The deodorant composition for sulfides according to claim 10, wherein the polyphenol is at least one species selected from the group consisting of a raw coffee bean extract, a tea extract and gallic acid.
